SEQUENCE LISTING (131 sequences)

SEQ ID NO: 1 - DNA, 41 bp, Sus scrofa
  misc_feature (21)..(21): n is A or C.
aaaatacattctcagagttcncattgtggcgcagcggaaac

SEQ ID NO: 2 - DNA, 41 bp, Sus scrofa
  misc_feature (21)..(21): n is T or C.
accgaccactagtcgccaccntctctctccatttcatttcg

SEQ ID NO: 3 - DNA, 41 bp, Sus scrofa
  misc_feature (21)..(21): n is A or G.
taaagatacaatatacttttntcatcattcagaataatgca

SEQ ID NO: 4 - DNA, 41 bp, Sus scrofa
  misc_feature (21)..(21): n is G or T.
tcactaactacaggtgctganaggtgctttcggttgttgtt

SEQ ID NO: 5 - DNA, 41 bp, Sus scrofa
  misc_feature (21)..(21): n is A or G.
tttctctgatgagattttaantacctacagagcacagtcct

SEQ ID NO: 6 - DNA, 41 bp, Sus scrofa
  misc_feature (21)..(21): n is T or A.
aaatacagacttgaagaccanttgaagcaaatgcatacttt

SEQ ID NO: 7 - DNA, 41 bp, Sus scrofa
  misc_feature (21)..(21): n is T or C.
tctctccacgctgagtaaatnttggtccaagaaggccaaac

SEQ ID NO: 8 - DNA, 41 bp, Sus scrofa
  misc_feature (21)..(21): n is T or C.
ctgtgagctgtggtgtgggtngcagacgcggctcggatccc

SEQ ID NO: 9 - DNA, 41 bp, Sus scrofa
  misc_feature (21)..(21): n is A or G.
gtgggtcgcagacgcggctcngatcccgtgttgctgtggct

SEQ ID NO: 10 - DNA, 41 bp, Sus scrofa
  misc_feature (21)..(21): n is C or T.
cagacgcggctcggatcccgngttgctgtggctctggcgta

SEQ ID NO: 11 - DNA, 41 bp, Sus scrofa
  misc_feature (21)..(21): n is G or A.
agccggggggggggggaggtncttttcaaagcacagagaaa

SEQ ID NO: 12 - DNA, 41 bp, Sus scrofa
  misc_feature (21)..(21): n is G or A.
cctaaaattaagagtgataanctagaggaacctacaaaaac

SEQ ID NO: 13 - DNA, 41 bp, Sus scrofa
  misc_feature (21)..(21): n is A or T.
tttgggcctgttctgttgttnaaatacataactgaaggagt

SEQ ID NO: 14 - DNA, 41 bp, Sus scrofa
  misc_feature (21)..(21): n is C or T.
ttttaagcaagggactaacanaattgaatttgtttggagag

SEQ ID NO: 15 - DNA, 41 bp, Sus scrofa
  misc_feature (21)..(21): n is G or A.
ataggatccccagcatacttntcatcagcatcctcctcctc

SEQ ID NO: 16 - DNA, 41 bp, Sus scrofa
  misc_feature (21)..(21): n is G or A.
tgtgccacagcagtgacccangcagctgcagtgacaatgcc

SEQ ID NO: 17 - DNA, 41 bp, Sus scrofa
  misc_feature: n is C or T.
tcaacgagctcaactagaatncatgaggatgtgggtttgct

SEQ ID NO: 18 - DNA, 41 bp, Sus scrofa
  misc_feature (21)..(21): n is G or A.
attgcagtggctaaggcgtcnggcagctgcagctccaattc

SEQ ID NO: 19 - DNA, 41 bp, Sus scrofa
  misc_feature (21)..(21): n is C or A.
gcaggtgtggccctaaaatgnaaaaaagaaaaagaaaagaa

SEQ ID NO: 20 - DNA, 41 bp, Sus scrofa
  misc_feature (21)..(21): n is G or T.
catgtcaataactattttatntatttatttatttatttatt

SEQ ID NO: 21 - DNA, 41 bp, Sus scrofa
  misc_feature (21)..(21): n is T or C.
ccatatgctgcagaggcagcngaaaaagaaaaaaattataa

SEQ ID NO: 22 - DNA, 41 bp, Sus scrofa
  misc_feature (21)..(21): n is G or A.
ggttcattcttgtagaggaanttactatggtaggtggaaaa

SEQ ID NO: 23 - DNA, 41 bp, Sus scrofa
  misc_feature (21)..(21): n is T or C.
gtaggtggaaaaacaagagcngacatttgagtttctttttt

SEQ ID NO: 24 - DNA, 41 bp, Sus scrofa
  misc_feature (21)..(21): n is G or C.
agaggggcgtggctaggagcngggatagggggaacccggtg

SEQ ID NO: 25 - DNA, 41 bp, Sus scrofa
  misc_feature (21)..(21): n is A or G.
gccacagcaacgcaggatccnagccaagtctgcaacctaca

SEQ ID NO: 26 - DNA, 41 bp, Sus scrofa
  misc_feature (21)..(21): n is C or T.
cgtgaatggagatagattttnggttttagaaaaaccccaga

SEQ ID NO: 27 - DNA, 41 bp, Sus scrofa
  misc_feature (21)..(21): n is G or A.
ctgggaattttatatgcagcngggcagccaaaaaaaaaaaa

SEQ ID NO: 28 - DNA, 41 bp, Sus scrofa
  misc_feature (21)..(21): n is T or C.
attgaagtttgtggcaatccnatgtcaaacaaatgtattgg

SEQ ID NO: 29 - DNA, 41 bp, Sus scrofa
  misc_feature (21)..(21): n is A or T.
tatatattatcacatacctgngtcatgttaactccctgtga

SEQ ID NO: 30 - DNA, 41 bp, Sus scrofa
  misc_feature (21)..(21): n is T or C.
accactggtggcaggtggatngactacaagcctccgaggtg

SEQ ID NO: 31 - DNA, 41 bp, Sus scrofa
  misc_feature (21)..(21): n is C or T.
cttcaatgttacatatttaantccagacatttgtaaacaca

SEQ ID NO: 32 - DNA, 41 bp, Sus scrofa
  misc_feature (21)..(21): n is A or G.
gattcactctgtggcactgtntccggtccctctgcccctcc

SEQ ID NO: 33 - DNA, 41 bp, Sus scrofa
  misc_feature (21)..(21): n is G or A.
ttctttcacagccttgtgagnaaggaaagccttttcccttg

SEQ ID NO: 34 - DNA, 41 bp, Sus scrofa
  misc_feature (21)..(21): n is G or A.
tctcaaaataaatctctagangatcccattactaacaatat

SEQ ID NO: 35 - DNA, 41 bp, Sus scrofa
  misc_feature (21)..(21): n is A or G.
agcctcctgatgctaaaatgnaagatcctataatgataaat

SEQ ID NO: 36 - DNA, 41 bp, Sus scrofa
  misc_feature (21)..(21): n is C or T.
ggaaaaataagaatctgtgantcaacagagtgaaaattaca

SEQ ID NO: 37 - DNA, 41 bp, Sus scrofa
  misc_feature (21)..(21): n is G or A.
ataacaaatttataaaacaantttctaattatcagggaaat

SEQ ID NO: 38 - DNA, 41 bp, Sus scrofa
  misc_feature (21)..(21): n is C or T.
gcataggccagcagcttcagntcctatttgacctctagcca

SEQ ID NO: 39 - DNA, 41 bp, Sus scrofa
  misc_feature (21)..(21): n is C or T.
gacctctagccagggaacctncatatgatgtggatacggcc

SEQ ID NO: 40 - DNA, 41 bp, Sus scrofa
  misc_feature (21)..(21): n is G or A.
gaatctgagcccagaggctgnaagcacacccttcctaagtg

SEQ ID NO: 41 - DNA, 41 bp, Sus scrofa
  misc_feature (21)..(21): n is G or A.
atctgagcccagaggctgaangcacacccttcctaagtggt

SEQ ID NO: 42 - DNA, 41 bp, Sus scrofa
  misc_feature (21)..(21): n is T or C.
tgaggttgcaggttcgatccntggccttcctcagtgggtta

SEQ ID NO: 43 - DNA, 41 bp, Sus scrofa
  misc_feature (21)..(21): n is A or G.
cctgggaacctccgtgtgccncaagtgcggccctaaaaagg

SEQ ID NO: 44 - DNA, 41 bp, Sus scrofa
  misc_feature (21)..(21): n is C or T.
acgttgctgtggctctggcgnaggctagtggctacagctcc

SEQ ID NO: 45 - DNA, 41 bp, Sus scrofa
  misc_feature (21)..(21): n is T or C.
ctgggaacctccatatgccgngggagcagctcaagaaaaga

SEQ ID NO: 46 - DNA, 41 bp, Sus scrofa
  misc_feature (21)..(21): n is C or T.
ccataaaatttcgtctgacanctagagaattctgaagtgag

SEQ ID NO: 47 - DNA, 41 bp, Sus scrofa
  misc_feature (21)..(21): n is A or G.
aaacctaagagagagaaaggnggaaaaccaaagagccttgt

SEQ ID NO: 48 - DNA, 41 bp, Sus scrofa
  misc_feature (21)..(21): n is C or T.
gccaggcactaagtcattcantgttaccatgtaactgtcag

SEQ ID NO: 49 - DNA, 41 bp, Sus scrofa
  misc_feature (21)..(21): n is A or C.
gttatgcaagcaaagagtagnagattaaattgtagtcagaa

SEQ ID NO: 50 - DNA, 41 bp, Sus scrofa
  misc_feature (21)..(21): n is G or A.
agcaaagagtagcagattaanttgtagtcagaagaatataa

SEQ ID NO: 51 - DNA, 41 bp, Sus scrofa
  misc_feature (21)..(21): n is C or A.
gggagttccctttgtggctcngtggttaacgaacccaacta

SEQ ID NO: 52 - DNA, 41 bp, Sus scrofa
  misc_feature (21)..(21): n is T or C.
aactaggatccatgaggatgngggttcaatccctggcctca

SEQ ID NO: 53 - DNA, 41 bp, Sus scrofa
  misc_feature (21)..(21): n is T or A.
ggttcctcaacaaactaaaantagaactaccgtatgatcca

SEQ ID NO: 54 - DNA, 41 bp, Sus scrofa
  misc_feature (21)..(21): n is A or C.
tccagagaaaaccataaatcnaaaagatacatgcaccctta

SEQ ID NO: 55 - DNA, 41 bp, Sus scrofa
  misc_feature (21)..(21): n is G or A.
acctaaatatccatcaatagnggaatggaagatgtggtaca

SEQ ID NO: 56 - DNA, 41 bp, Sus scrofa
  misc_feature (21)..(21): n is G or A.
tttactctgttgtacacctgnaactaatacaatattataaa

SEQ ID NO: 57 - DNA, 41 bp, Sus scrofa
  misc_feature (21)..(21): n is T or C.
ctttcaaattcccagtaattntgaattttaaacatgaaaaa

SEQ ID NO: 58 - DNA, 41 bp, Sus scrofa
  misc_feature (21)..(21): n is C or G.
tataatcctatgaattttggnttgcagagtcagagagaagt

SEQ ID NO: 59 - DNA, 41 bp, Sus scrofa
  misc_feature (21)..(21): n is G or C.
gcacggactcaggcctgagangacccatgcaggtggtgggt

SEQ ID NO: 60 - DNA, 41 bp, Sus scrofa
  misc_feature (21)..(21): n is G or A.
tttgtggaaacaagggtctcnagtacactgcccagattttt

SEQ ID NO: 61 - DNA, 41 bp, Sus scrofa
  misc_feature (21)..(21): n is T or A.
tggtgggatatcggagcagcnagatccaggtgtcagagact

SEQ ID NO: 62 - DNA, 41 bp, Sus scrofa
  misc_feature (21)..(21): n is T or C.
ggcacagccagtgctcccccngccccctcctccccagggtg

SEQ ID NO: 63 - DNA, 41 bp, Sus scrofa
  misc_feature (21)..(21): n is A or G.
cagtgggctaaggatccggcnttgccgtgagctgtggtgta

SEQ ID NO: 64 - DNA, 41 bp, Sus scrofa
  misc_feature (21)..(21): n is T or C.
ggctaaggatccggcgttgcngtgagctgtggtgtaggtcg

SEQ ID NO: 65 - DNA, 41 bp, Sus scrofa
  misc_feature (21)..(21): n is C or T.
gtggctcggatcccgcgttgnggtggctctggcgtaggccg

SEQ ID NO: 66 - DNA, 41 bp, Sus scrofa
  misc_feature (21)..(21): n is T or C.
gctctcaaagactgtgaggcnggaagacaaactgacagttt

SEQ ID NO: 67 - DNA, 41 bp, Sus scrofa
  misc_feature (21)..(21): n is A or G.
tctgcaccagagagagagggnagccacatgttacaacacta

SEQ ID NO: 68 - DNA, 41 bp, Sus scrofa
  misc_feature (21)..(21): n is G or T.
aaccaggataaaaggacattnactgcaaaacacggtgtctg

SEQ ID NO: 69 - DNA, 41 bp, Sus scrofa
  misc_feature (21)..(21): n is A or C.
gctctcgagtcacgaatctanacaaaatcacctctctgaaa

SEQ ID NO: 70 - DNA, 41 bp, Sus scrofa
  misc_feature (21)..(21): n is C or T.
cggctcggatcctgtgttgcngtggctctggcgtaggccag

SEQ ID NO: 71 - DNA, 41 bp, Sus scrofa
  misc_feature (21)..(21): n is A or G.
ccctggtacccacaagacccngagcttccttcccgtcactg

SEQ ID NO: 72 - DNA, 41 bp, Sus scrofa
  misc_feature (21)..(21): n is T or C.
gaagtcaattcttcaaccatnctaaggaaataaattaccta

SEQ ID NO: 73 - DNA, 41 bp, Sus scrofa
  misc_feature (21)..(21): n is C or A.
ctgaggggactcaggaaaccntatcaacagagtgattccac

SEQ ID NO: 74 - DNA, 41 bp, Sus scrofa
  misc_feature (21)..(21): n is A or G.
gcctccacatttacttactcnattaaccagtcaatagctct

SEQ ID NO: 75 - DNA, 41 bp, Sus scrofa
  misc_feature (21)..(21): n is T or C.
ttttgccaatgaaaactcttnccccaaaccatggcggagtt

SEQ ID NO: 76 - DNA, 41 bp, Sus scrofa
  misc_feature (21)..(21): n is C or G.
gaacactcccaggtcaccagnctccattctgacactgtgtc

SEQ ID NO: 77 - DNA, 41 bp, Sus scrofa
  misc_feature (21)..(21): n is A or G.
atgcggggcactaggaagccngtgttcatggggcactgggc

SEQ ID NO: 78 - DNA, 41 bp, Sus scrofa
  misc_feature (21)..(21): n is A or G.
aacaaaggcagcatggggganggggcagagtggagcatggg

SEQ ID NO: 79 - DNA, 41 bp, Sus scrofa
  misc_feature (21)..(21): n is T or C.
ttcaacccctagcctgggaanctccatatgctgcaggagcg

SEQ ID NO: 80 - DNA, 41 bp, Sus scrofa
  misc_feature (21)..(21): n is C or A.
aaaagaaagaccggaaaaaanaaacaaacaaaccaaaaatc

SEQ ID NO: 81 - DNA, 41 bp, Sus scrofa
  misc_feature (21)..(21): n is C or T.
ggctggttcaactgaggaacngaattcttaatttctgtcaa

SEQ ID NO: 82 - DNA, 41 bp, Sus scrofa
  misc_feature (21)..(21): n is A or C.
ggttcctagtcggattcgttnaccactgcaccacgatggga

SEQ ID NO: 83 - DNA, 41 bp, Sus scrofa
  misc_feature (21)..(21): n is C or T.
cgttcaccactgcaccacgangggaactccttatttgcttt

SEQ ID NO: 84 - DNA, 41 bp, Sus scrofa
  misc_feature (21)..(21): n is G or T.
gcaccacgatgggaactcctnatttgctttattaaatgatt

SEQ ID NO: 85 - DNA, 41 bp, Sus scrofa
  misc_feature (21)..(21): n is A or C.
ccgcgcgacgtgaggggttcnccgcccgggcgcttgtttcc

SEQ ID NO: 86 - DNA, 41 bp, Sus scrofa
  misc_feature (21)..(21): n is T or C.
cgagctctatgttggcttctnaggctacctgccgagcattt

SEQ ID NO: 87 - DNA, 41 bp, Sus scrofa
  misc_feature (21)..(21): n is T or A.
gaaatggcaaaagctaagtcntggagttatgtttctaataa

SEQ ID NO: 88 - DNA, 41 bp, Sus scrofa
  misc_feature (21)..(21): n is T or C.
cctgaagcccattctgagtanaactgccgagtgtaactgac

SEQ ID NO: 89 - DNA, 41 bp, Sus scrofa
  misc_feature (21)..(21): n is A or G.
agactcatatcaaaacttcanaataactacaaaccaaaaat

SEQ ID NO: 90 - DNA, 41 bp, Sus scrofa
  misc_feature (21)..(21): n is C or T.
ccaatgactgttcagtactangtgtggaaatgacgcaagga

SEQ ID NO: 91 - DNA, 41 bp, Sus scrofa
  misc_feature (21)..(21): n is A or G.
aaaaacagacacagatcaatngaacagagagctcagaaaga

SEQ ID NO: 92 - DNA, 41 bp, Sus scrofa
  misc_feature (21)..(21): n is T or C.
ctctgaacaaggccagggatngaacccacaacctcatggtt

SEQ ID NO: 93 - DNA, 41 bp, Sus scrofa
  misc_feature (21)..(21): n is C or A.
cttccaagatttaaccactangtgtaatatttgctttatgt

SEQ ID NO: 94 - DNA, 41 bp, Sus scrofa
  misc_feature (21)..(21): n is T or G.
tacaccagagccacagcaacncgggatccgagccgagtctg

SEQ ID NO: 95 - DNA, 41 bp, Sus scrofa
  misc_feature (21)..(21): n is G or A.
tatactagtttacatctgctnatcccaaactcccagtctgc

SEQ ID NO: 96 - DNA, 41 bp, Sus scrofa
  misc_feature (21)..(21): n is A or T.
actgcaaatggcattatttcnttttttttttttatggctgg

SEQ ID NO: 97 - DNA, 41 bp, Sus scrofa
  misc_feature (21)..(21): n is T or C.
tatttatgtaccacatcttcnatatctatttctctgtctat

SEQ ID NO: 98 - DNA, 41 bp, Sus scrofa
  misc_feature (21)..(21): n is C or A.
tggctgcaccagtttacattncttctattagcatagaaggg

SEQ ID NO: 99 - DNA, 41 bp, Sus scrofa
  misc_feature (21)..(21): n is  C or T.
ttacattacttctattagcanagaagggtccccttttgtct

SEQ ID NO: 100 - DNA, 41 bp, Sus scrofa
  misc_feature (21)..(21): n is T or C.
agctgcatttccttaaactcntgtggtgctcagaaaagtct

SEQ ID NO: 101 - DNA, 41 bp, Sus scrofa
  misc_feature (21)..(21): n is C or G.
gggtgatgtgggtgtgggctngggtgggtgtcccaggaggg

SEQ ID NO: 102 - DNA, 41 bp, Sus scrofa
  misc_feature (21)..(21): n is A or G.
ggagggattggaatggactangaatttggggtcaatagatg

SEQ ID NO: 103 - DNA, 41 bp, Sus scrofa
  misc_feature (21)..(21): n is A or C.
aggccacgccacatgctgganctggttgcgaacatgggtct

SEQ ID NO: 104 - DNA, 41 bp, Sus scrofa
  misc_feature (21)..(21): n is A or T.
ggaacaatgaggttgcgggtncaatccctgcccttgctcac

SEQ ID NO: 105 - DNA, 41 bp, Sus scrofa
  misc_feature (21)..(21): n is A or G.
ggcagagatttgcaagagcanttggacaaacagaactgcat

SEQ ID NO: 106 - DNA, 41 bp, Sus scrofa
  misc_feature (21)..(21): n is T or C.
ccttggtgagtttgtccatcnccaaacattgctacctttgg

SEQ ID NO: 107 - DNA, 41 bp, Sus scrofa
  misc_feature (21)..(21): n is T or C.
gtaactagcagtttggagctngggcaaaggagaacaaaaca

SEQ ID NO: 108 - DNA, 41 bp, Sus scrofa
  misc_feature (21)..(21): n is T or G.
ttatatttctggaagaagcanaaagatgactatcagacaca

SEQ ID NO: 109 - DNA, 41 bp, Sus scrofa
  misc_feature (21)..(21): n is T or C.
ttttatacttctttccactcnctgactttcagtacatgata

SEQ ID NO: 110 - DNA, 41 bp, Sus scrofa
  misc_feature (21)..(21): n is T or C.
ttcctttttctttttagaacngcactttcggcacgtggaag

SEQ ID NO: 111 - DNA, 41 bp, Sus scrofa
  misc_feature (21)..(21): n is C or T.
ttagaactgcactttcggcangtggaagtttccaggctagg

SEQ ID NO: 112 - DNA, 41 bp, Sus scrofa
  misc_feature (21)..(21): n is T or C.
gcaaacgtccgtactaccgcnagagtgttggaagactgaat

SEQ ID NO: 113 - DNA, 41 bp, Sus scrofa
  misc_feature (21)..(21): n is T or C.
ttcttgattgctctcagatcnatcctttcccaaatttgcac

SEQ ID NO: 114 - DNA, 41 bp, Sus scrofa
  misc_feature (21)..(21): n is A or G.
caaacacctactgagcacctnccccatgacgagcactgttg

SEQ ID NO: 115 - DNA, 41 bp, Sus scrofa
  misc_feature (21)..(21): n is A or G.
aggatcagaaaaggtccaaancagcacagagacaccaacat

SEQ ID NO: 116 - DNA, 41 bp, Sus scrofa
  misc_feature (21)..(21): n is T or A.
cctaaaatatgaaatcatacntgtaaattaactgagaacat

SEQ ID NO: 117 - DNA, 41 bp, Sus scrofa
  misc_feature (21)..(21): n is A or G.
acctgcactagagccgtgacnccagatccttaaccactagg

SEQ ID NO: 118 - DNA, 41 bp, Sus scrofa
  misc_feature (21)..(21): n is T or C.
aggccaaggctgcatttcacnaacctgcctcgatgtccttg

SEQ ID NO: 119 - DNA, 41 bp, Sus scrofa
  misc_feature (21)..(21): n is T or C.
tgccctcagagaaacaccatngcacaacagcttctagggag

SEQ ID NO: 120 - DNA, 41 bp, Sus scrofa
  misc_feature (21)..(21): n is G or A.
gtgcatgttaagtgagcggtnctggtctaaagcactgtgta

SEQ ID NO: 121 - DNA, 41 bp, Sus scrofa
  misc_feature (21)..(21): n is G or C.
gcaggatgtgtagacctgganaggccctgagcaacccatga

SEQ ID NO: 122 - DNA, 41 bp, Sus scrofa
  misc_feature (21)..(21): n is A or G.
caactctggaggctcaaacantagtgtgagacagctggaac

SEQ ID NO: 123 - DNA, 41 bp, Sus scrofa
  misc_feature (21)..(21): n is C or T.
ggggtctttggacccagctangggttagagagcaataggct

SEQ ID NO: 124 - DNA, 41 bp, Sus scrofa
  misc_feature (21)..(21): n is T or C.
tttgaaaaagaagccctggcnacacttgcctgtgactacgc

SEQ ID NO: 125 - DNA, 41 bp, Sus scrofa
  misc_feature (21)..(21): n is C or T.
ccacccacctggctccctaantgtgtctttatttttatttt

SEQ ID NO: 126 - DNA, 41 bp, Sus scrofa
  misc_feature (21)..(21): n is G or A.
ccatttctactaaaaaaggangttcagaggaactcagggac

SEQ ID NO: 127 - DNA, 41 bp, Sus scrofa
  misc_feature (21)..(21): n is G or A.
aatgaatgggagatggagttntagtagaagagtgtgtttga

SEQ ID NO: 128 - DNA, 41 bp, Sus scrofa
  misc_feature (21)..(21): n is G or A.
gaaccatgaggtgcaggttcnatccctggcctcgctcagtg

SEQ ID NO: 129 - DNA, 41 bp, Sus scrofa
  misc_feature (21)..(21): n is A or G.
cacggtgctgtggctctggcnaaggcggcagctacagctcc

SEQ ID NO: 130 - DNA, 41 bp, Sus scrofa
  misc_feature (21)..(21): n is G or A.
tccttttgccaattcccctantaaatctttctcttgtgtat

SEQ ID NO: 131 - DNA, 41 bp, Sus scrofa
  misc_feature (21)..(21): n is T or C.
tggaccaccaggaaactcccntgtatataagtttttaattt

(19) United States
(12) Patent Application Publication
(10) Pub. No.: US 2017/0159122 A1
(43) Pub. Date: Jun. 8, 2017

(54) METHODS AND KITS FOR DETECTING GENETIC MARKERS FOR LITTER SIZE IN PIGS

(71) Applicants: Kunming Institute of Zoology, Chinese Acadermy of Sciences, Yunnan (CN); Jiangxi Agricultural University, Jiangxi (CN)

(72) Inventors: Yaping ZHANG, Yunnan (CN); Lusheng HUANG, Jiangxi (CN); Haibing XIE, Yunnan (CN); Jun REN, Jiangxi (CN); Huashui AI, Jiangxi (CN)

(21) Appl. No.: 15/129,830

(22) PCT Filed: Mar. 28, 2014
(86) PCT No.: PCT/CN2014/074247

§ 371 Date: Sep. 28, 2016

(51) Int. Cl. C12Q 1/68

(52) U.S. Cl. CPC C12Q 1/6883; C12Q 2600/124; C12Q 2600/156

(57) ABSTRACT

Methods and kits useful for identifying a pig as producing an increased litter size are provided. Genetic makers, in particular SNP markers are provided that are useful in distinguishing pigs for a phenotype associated with increased litter size.

### BACKGROUND

Pig is a world-wide economic animal and is an important meat source. In 2009, about 966 million pigs were raised in the world, the approximate value of the pig meat production reached 163 billion US dollars, and near half (476 million) of the pigs were in China (http://www.fao.org). The population size has increased annually with the increasing global market demands. It is critical to raise a population of sows and boars proportional to the total population to produce piglets. During the pig reproduction, the number of boars is much less than that of sows. Generally, the ratio of population size of sows to the size of total population is slightly larger than 1/10, which amounts to a quite large sow population with a size about 100 million in the world.

Usually, the cost of pig production is mainly from the feed. For pig reproduction, special cares are required during the pregnant and lactation period, which increase the cost of raising sows. Reducing the sow population size but still capable of providing enough piglets would benefit the swine industry. 10% reduction of the sow population size would produce savings of the cost in raising 10 million sows per year, which can be achieved by producing 1 extra piglet per litter for sows. The improvement of the reproductive efficiency, reflected as litter size of sows, is an economic concern in the swine industry.

Chinese Taihu pigs consist of several local breeds (including Erhualian, Meishan, and Fengjing pigs, etc.) from geographically closely-linked regions of Taihu Basin. Taihu pigs are well known for their prolificacy; for example, Taihu pigs can produce about 4-5 extra piglets per litter than most of the other Chinese local breeds and western commercial breeds [1]. The genetic sequences/variants underlying the large litter size of Taihu pigs are valuable resources in improving the litter size trait of pigs.

Genetic markers associated with litter size have been well described in several genes, such as OPN (U.S. Pat. No. 6,410,227B1), PRLR (U.S. Pat. No. 7,081,335B2), FSHβ(U.S. Pat. No. 6,291,174B1), ESR (U.S. Pat. No. 5,550,024A), and RBP4 [2]. Quantitative trait loci (QTL) mappings have identified many regions responsible for the large litter size in Taihu pigs, spanning chromosomes 6, 7, 8 and 15 [3,4]. However, the QTLs usually span through large genomic regions, and the genetic variants are not well resolved. Identification of the genetic variants underlying the difference of reproductive capability between Taihu pigs and other pigs has utility in breeding sows with much higher efficiency. Therefore, it is highly desired to find the genomic regions that are artificially selected in Erhualian pigs, and to identify the genetic variants of large litter size that can be used in the molecular breeding of pigs.

### SUMMARY OF THE INVENTION

One aspect of the invention relates to methods of identifying a pig as producing an increased litter size comprising detecting for presence or absence of an E allele SNP marker in at least one single nucleotide polymorphism (SNP) site within a genomic region selected from the group consisting of Sweep A, Sweep B, Sweep C, Sweep D, and Sweep E, in a pig genome, wherein the presence of the E allele SNP marker indicates an increased litter size of the pig.

In certain embodiments, the genomic region is Sweep A. In these embodiments, the methods further comprise detecting for presence or absence of the E allele SNP marker in the SNP site of one or both copies of the chromosomes. In certain embodiments, the presence of the E allele SNP marker in the SNP site of one or both copies of chromosomes indicates an increased litter size of the pig.

In certain embodiments, the Sweep A genomic region spans from Chr6: 122097788 to Chr6: 122217096 (NCBI build Sscrofa 10.2). In certain embodiments, the at least one SNP site within Sweep A genomic region is selected from the SNPs: A1-A13 as listed in Table 1. In certain embodiments, the at least one SNP site within Sweep A genomic region comprises SNP A2, or is in linkage disequilibrium with SNP A2.

In certain embodiments, the genomic region is Sweep B. In these embodiments, the methods further comprise detecting for presence or absence of the E allele SNP marker in the SNP site of both copies of the chromosomes. In certain embodiments, the presence of the E allele SNP marker in the SNP site of both copies of chromosomes indicates an increased litter size of the pig.

In certain embodiments, the Sweep B genomic region spans from Chr6: 89403626 to Chr6: 90311616 (NCBI build Sscrofa 10.2). In certain embodiments, the at least one SNP site within Sweep B genomic region is selected from the SNPs: B1-B91 as listed in Table 1. In certain embodiments, the at least one SNP site within Sweep B genomic region comprises SNP B13, or is in linkage disequilibrium with SNP B13.

In certain embodiments, the genomic region is Sweep C. In these embodiments, the methods further comprise detecting for presence or absence of the E allele SNP marker in the SNP site of one or both copies of the chromosomes. In certain embodiments, the presence of the E allele SNP marker in the SNP site of one or both copies of chromosomes indicates an increased litter size of the pig.

In certain embodiments, the Sweep C genomic region locates at Chr7: 63714553 (NCBI build Sscrofa 10.2). In certain embodiments, the SNP site within Sweep C genomic region is SNP C1 as listed in Table 1.

In certain embodiments, the genomic region is Sweep D. In these embodiments, the methods further comprise detecting for presence or absence of the E allele SNP marker in the SNP site of both copies of the chromosomes. In certain embodiments, the presence of the E allele SNP marker in the SNP site of one copy of chromosomes indicates an increased litter size of the pig.

In certain embodiments, the Sweep D genomic region spans from Chr15: 51799437 to Chr15: 51800356 (NCBI build Sscrofa 10.2). In certain embodiments, the at least one SNP site within Sweep D genomic region is selected from the SNPs: D1-D3 as listed in Table 1. In certain embodiments, the at least one SNP site within Sweep D genomic region comprises SNP D1, or is in linkage disequilibrium with SNP D1.

In certain embodiments, the genomic region is Sweep E. In these embodiments, the methods further comprise detecting for presence or absence of the E allele SNP marker in the SNP site of one or both copies of the chromosomes. In certain embodiments, the presence of the E allele SNP marker in the SNP site of one or both copies of chromosomes indicates an increased litter size of the pig.

In certain embodiments, the Sweep E genomic region spans from Chr3: 72655441 to Chr3: 72795872 (NCBI build Sscrofa 10.2). In certain embodiments, the at least one SNP site within Sweep E genomic region is selected from the SNPs: E1-E23 as listed in Table 1. In certain embodiments, the at least one SNP site within Sweep E genomic region comprises SNP E10, or is in linkage disequilibrium with SNP E10.

In certain embodiments, the E allele SNP marker is the high-frequency nucleotide at the corresponding SNP site in the genomes of Erhualian pigs. In certain embodiments, the E allele SNP markers are as shown in Table 1.

In certain embodiments, the methods further comprise selecting the pig for breeding if the pig is identified as producing an increased litter size.

In certain embodiments, the methods further comprise obtaining a germ cell from the pig for breeding if the pig is identified as producing an increased litter size.

Any pig can be tested using the methods disclosed herein. In certain embodiments, the pig is a sow, an offspring of a Taihu pig, or an offspring of an Erhualian pig.

Any suitable methods can be used to conduct the detection described herein. In certain embodiments, the detecting comprises sequencing at least a fragment containing the SNP site in a nucleic acid sample from the pig. In certain embodiments, the detecting comprises detecting an amplification product of at least a fragment containing the SNP site in a nucleic acid sample from the pig. In certain embodiments, the detecting comprises detecting hybridization of a probe to at least a fragment containing the SNP site in a nucleic acid sample from the pig. In certain embodiments, the detecting comprises detecting a primer extension product of at least a fragment containing the SNP site in a nucleic acid sample from the pig. In certain embodiments, the detecting comprises detecting restriction digestion product of at least a fragment containing the SNP site in a nucleic acid sample from the pig. In certain embodiments, the detecting comprises detecting gel electrophoresis results of at least a fragment containing the SNP site in a nucleic acid sample from the pig. In certain embodiments, the detecting comprises detecting binding affinity of a protein to at least a fragment containing the SNP site in a nucleic acid sample from the pig.

In another aspect, the present disclosure provides methods of detecting the SNP marker at a SNP site within Sweep A region in a pig, comprising: determining the presence or absence of an E allele SNP marker or an O allele SNP marker in at least one single nucleotide allele (SNP) site within Sweep A genomic region in a pig genome. In certain embodiments, the Sweep A genomic region spans from Chr6: 122097788 to Chr6: 122217096 (NCBI build Sscrofa 10.2). In certain embodiments, the at least one SNP site within Sweep A genomic region is selected from the SNPs: A1-A13 as listed in Table 1. In certain embodiments, the at least one SNP site within Sweep A genomic region comprises SNP A2, or is in linkage disequilibrium with SNP A2.

In another aspect, the present disclosure provides methods of detecting the SNP marker at a SNP site within Sweep B region in a pig, comprising: determining the presence or absence of an E allele SNP marker or an O allele SNP marker in at least one single nucleotide allele (SNP) site within Sweep B genomic region in a pig genome. In certain embodiments, the Sweep B genomic region spans from Chr6: 89403626 to Chr6: 90311616 (NCBI build Sscrofa 10.2). In certain embodiments, the at least one SNP site within Sweep B genomic region is selected from the SNPs: B1-B91 as listed in Table 1. In certain embodiments, the at least one SNP site within Sweep B genomic region comprises SNP B13, or is in linkage disequilibrium with SNP B13.

In another aspect, the present disclosure provides methods of detecting the SNP marker at a SNP site within Sweep C region in a pig, comprising: determining the presence or absence of an E allele SNP marker or an O allele SNP marker in the single nucleotide allele (SNP) site within Sweep C genomic region in a pig genome. In certain embodiments, the Sweep C genomic region locates at Chr7: 63714553 (NCBI build Sscrofa 10.2). In certain embodiments, the SNP site within Sweep C genomic region is the SNP C1 as listed in Table 1.

In another aspect, the present disclosure provides methods of detecting the SNP marker at a SNP site within Sweep D region in a pig, comprising: determining the presence or absence of an E allele SNP marker or an O allele SNP marker in at least one single nucleotide allele (SNP) site within Sweep D genomic region in a pig genome. In certain embodiments, the Sweep D genomic region spans from Chr15: 51799437 to Chr15: 51800356 (NCBI build Sscrofa 10.2). In certain embodiments, the at least one SNP site within Sweep D genomic region is selected from the SNPs: D1-D3 as listed in Table 1. In certain embodiments, the at least one SNP site within Sweep D genomic region comprises SNP D1, or is in linkage disequilibrium with SNP D1.

In another aspect, the present disclosure provides methods of detecting the SNP marker at a SNP site within Sweep E region in a pig, comprising: determining the presence or absence of an E allele SNP marker or an O allele SNP marker in at least one single nucleotide allele (SNP) site within Sweep E genomic region in a pig genome. In certain embodiments, the Sweep E genomic region spans from Chr3: 72655441 to Chr3: 72795872 (NCBI build Sscrofa 10.2). In certain embodiments, the at least one SNP site within Sweep E genomic region is selected from the SNPs: E1-E23 as listed in Table 1. In certain embodiments, the at least one SNP site within Sweep E genomic region comprises SNP E10, or is in linkage disequilibrium with SNP E10.

In another aspect, the present disclosure provides isolated oligonucleotide primers, which are useful in selectively amplifying a polynucleotide fragment containing an E allele SNP marker in at least one of the SNP sites listed in Table 1, or selectively amplifying a polynucleotide fragment lacking an E allele SNP marker (e.g. containing an O allele SNP marker) in at least one of the SNP sites listed in Table 1.

In another aspect, the present disclosure provides isolated oligonucleotide probes, which are useful in selectively hybridizing to a polynucleotide fragment containing an E allele SNP marker in one or more SNP sites listed in Table 1, or selectively hybridizing to a polynucleotide fragment lacking an E allele SNP marker (e.g. containing an O allele SNP marker) in one or more SNP sites listed in Table 1.

In another aspect, the present disclosure provides kits useful in the methods provided herein. The kits comprise the isolated oligonucleotide primers provided herein, or the isolated oligonucleotide probes provided herein.

### DESCRIPTION OF THE DRAWINGS

FIG. 1. The litter size of three genotypes examined using a tag SNP chr6:122,113,635 for individuals in a White Duroc×Erhualian F2 sow population. * indicates the difference with a statistical significance of p<0.05; and ** indicates the difference with a statistical significance of p<0.01.

FIG. 2. The litter size of three genotypes examined using a tag SNP chr6:122,113,635 for individuals in a Sutai sow population. * indicates the difference with a statistical significance of p<0.05.

FIG. 3. The litter size of three genotypes examined using a tag SNP chr6:89,899,151 for individuals in a White Duroc×Erhualian F2 sow population. * indicates the difference with a statistical significance of p<0.05.

FIG. 4. The litter size of three genotypes examined using a tag SNP chr7: 63,714,553 for individuals in a White Duroc×Erhualian F2 sow population. ** indicates the difference with a statistical significance of p<0.01.

FIG. 5. The litter size of three genotypes examined using a tag SNP chr7: 63,714,553 for individuals in a Sutai sow population. * indicates the difference with a statistical significance of p<0.05. ** indicates the difference with a statistical significance of p<0.01.

FIG. 6. The litter size of three genotypes examined using a tag SNP chr15:51,799,437 for individuals in a White Duroc×Erhualian F2 sow population. ** indicates the difference with a statistical significance of p<0.01.

FIG. 7. The litter size of three genotypes examined using a tag SNP chr15: 51,799,437 for individuals in a Sutai sow population. * indicates the difference with a statistical significance of p<0.05.

FIG. 8. The litter size of three genotypes examined using a tag SNP chr3:72,759,645 for individuals in a Large White sow population. ** indicates the difference with a statistical significance of p<0.01; and * indicates the difference with a statistical significance of p<0.05.

FIG. 9. The litter size of three genotypes examined using a tag SNP chr3:72,759,645 for individuals in a Large White×Landrace F1 crossbred sow population. ** indicates the difference with a statistical significance of p<0.01.

### DETAILED DESCRIPTION OF THE INVENTION

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

Genetic Markers for Increased Litter Size in Pig

In one aspect, the present disclosure provides genetic markers associated with an increased litter size in a pig.

The term “genetic marker” as used herein refers to, a nucleotide sequence on a known location of a genome or a chromosome. The genetic markers provided herein are associated with a phenotype involving litter size of a pig, for example, a phenotype of an increased litter size.

By “litter size,” it is meant the number of offspring at one birth of animals from the same mother. An increased litter size is a litter size larger than an average litter size for a pig population. For example, an increased litter size is at least 2%, at least 3%, at least 5%, at least 7%, at least 8%, at least 10%, at least 15%, at least 20%, at least 25%, or even more than an average litter size for a pig population. For another example, an increased litter size is at least 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5 or more pigs per litter than an average litter size produced by a pig population.

The term “genome” as used herein, encompasses the genomic DNA sequence from a pig somatic cell which has two copies of chromosomes, and also the genomic DNA sequence from a pig germ cell which has a single copy of chromosome.

In certain embodiments, the genetic marker provided herein is a single nucleotide polymorphism (SNP) marker. SNP is a single nucleotide variation occurring among a population at a particular location (i.e. the SNP site) in a genome or chromosome, in other words, different individuals may have different nucleotides at the particular SNP site, for example, some may have A at the SNP site while some others may have C at the site. The nucleotide at the SNP site is a SNP marker. For a given population, the SNP marker can vary among A, T, G, and C, each at an equal frequency or at a different frequencies, for example, certain SNP markers (e.g. A, or T) may be found at a high frequency which is above average probability, and certain other SNP markers (e.g. G, or C) may be found at a low frequency which is below the average probability. The frequency of each SNP marker at the SNP site may or may not be conserved among different populations. For example, for a given SNP site, a SNP marker is found at high frequency in population A, but such marker may or may not be found at a similarly high frequency in population B, or may be even found at low frequency in population B. Without being bound to any theory, it is contemplated that, in certain occasions, such difference in the high frequency SNP marker at a SNP site can be associated with difference in a phenotype in different populations. For example, assuming that: 1) population A has larger litter size than population B (i.e. different phenotype); and 2) population A is found having a high frequency SNP marker of “C” at a given SNP site, but population B is having a high frequency “T” at the given SNP site (i.e. different high frequency SNP markers); if the phenotype (large litter size) of population A can be correlated with the presence of the “C” at the SNP site (i.e. the high frequency SNP marker in population A), then the SNP marker at the SNP site can be used as the genetic marker for determining whether a candidate has a phenotype of large letter size.

In certain embodiments, the genetic markers provided herein relate to high frequency SNP markers found in the Erhualian pig genomes. The Erhualian pig is an indigenous breed of pigs distributed mainly in East China, see details in, Animal genetic resources in China: Pigs, published by China Agriculture press, p41-44, 2011. Erhualian pig is a well-known prolific breed, which produces larger litter size than other pig breeds. It is unexpectedly found by the inventors that, the phenotype of large litter size in Erhualian pigs is correlated with certain high frequency SNP markers at some SNP sites of Erhualian genome. In such SNP sites, the high frequency SNP markers in the Erhualian genome are different from those high frequency SNP markers in genomes from other pig breeds, such as pigs of Tibetan breed, Bama xiang breed, Laiwu breed, and Diannan small-ear pigs and wild boars, which are known to produce a smaller litter size than Erhualian breed. This make the SNP markers disclosed herein useful as a genetic marker for identifying pigs with a phenotype of large litter size.

In certain embodiments, the genetic markers provided herein are SNP markers within the Sweep A genomic region on chromosome 6 of a pig genome. Sweep A genomic region, as used herein, refers to the region which spans from Chr6: 122097788 to Chr6: 122217096 (NCBI build Sscrofa 10.2). In certain embodiments, the genetic markers provided herein in the Sweep A region are SNPs A1-A13, whose locations are summarized in Table 1. To better illustrate the location, Table 1 also shows the 20 bp sequences flanking each of the SNP site, so that people in the art may also find the SNP sites on a given pig genome by sequence homology search.

In certain embodiments, the genetic markers provided herein are SNP markers within the Sweep B genomic region on chromosome 6 of a pig genome. Sweep B genomic region, as used herein, refers to the region which spans from Chr6: 89403626 to Chr6: 90311616 (NCBI build Sscrofa 10.2). In certain embodiments, the genetic markers provided herein in the Sweep B region are SNPs B1-B91, whose locations are summarized in Table 1. To better illustrate the location, Table 1 also shows the 20 bp sequences flanking each of the SNP site, so that people in the art may also find the SNP sites on a given pig genome by sequence homology search.

In certain embodiments, the genetic marker provided herein is the SNP marker within the Sweep C genomic region on chromosome 7 of a pig genome. Sweep C genomic region, as used herein, refers to the nucleotide site which locates at Chr7: 63714553 (NCBI build Sscrofa 10.2). In certain embodiments, the genetic marker provided herein in the Sweep C region is SNP C1, whose location is summarized in Table 1. To better illustrate the location, Table 1 also shows the 20 bp sequences flanking each of the SNP site, so that people in the art may also find the SNP site on a given pig genome by sequence homology search.

In certain embodiments, the genetic markers provided herein are SNP markers within the Sweep D genomic region on chromosome 15 of a pig genome. Sweep D genomic region, as used herein, refers to the region which spans from Chr15: 51799437 to Chr15: 51800356 (NCBI build Sscrofa 10.2). In certain embodiments, the genetic markers provided herein in the Sweep D region are SNPs D1-D3, whose locations are summarized in Table 1. To better illustrate the location, Table 1 also shows the 20 bp sequences flanking each of the SNP site, so that people in the art may also find the SNP sites on a given pig genome by sequence homology search.

In certain embodiments, the genetic markers provided herein are SNP markers within the Sweep E genomic region on chromosome 3 of a pig genome. Sweep E genomic region, as used herein, refers to the region which spans from Chr3: 72655441 to Chr3: 72795872 (NCBI build Sscrofa 10.2). In certain embodiments, the genetic markers provided herein in the Sweep E region are SNPs E1-E23 whose locations are summarized in Table 1. To better illustrate the location, Table 1 also shows the 20 bp sequences flanking each of the SNP site, so that people in the art may also find the SNP sites on a given pig genome by sequence homology search.

For SNP sites A1-A13, B1-B91, C1, D1-D3, E1-E23, the high frequency SNP marker found in Erhualian pig genomes (i.e. E allele, stands for Erhualian allele, shown in Table 1) is different from that found in non-Erhualian pig genomes (i.e. O allele, stands for other pig allele, shown in Table 1). It has also been shown that, pigs with the E allele SNP marker is associated with a phenotype of large litter size, whereas pigs with the O allele SNP marker is associated with a relatively smaller litter size. By detecting the presence or absence of the E allele SNP markers, it allows identification of a pig with a phenotype of large litter size.

The SNP markers can be in linkage disequilibrium. The term “linkage disequilibrium” as used herein, refers to non-random association of more than one SNP markers. For example, for a given population, if SNP marker “A” at SNP site X occurs at a frequency of 50%, and SNP marker “T” at SNP site Y occurs at a frequency of 50%, then the presence of both SNP marker “A” at SNP site X and SNP marker “T” at SNP site Y in one individual is predicted to occur at a frequency of 25%. However, if the two SNP markers are found occurring together at a rate significantly higher than 25%, then the two SNP markers are tend to be transmitted together at a higher rate than what would be predicted based on their independent frequencies of occurrence. Therefore, SNP markers in linkage disequilibrium tend to be inherited or genetically passed to an offspring in a combined fashion (e.g. as if they are associated through a linkage). For example, if one of the SNP markers is passed to an offspring, it is likely that the other SNP markers in linkage disequilibrium with that marker are also inherited by the offspring. In other words, for SNP markers in linkage disequilibrium, presence of any one of these markers would indicate the likelihood of presence of the other markers. Accordingly, if presence of a SNP marker at a SNP site indicates a desirable phenotype (e.g. increased litter size), then presence of any other SNP genetic markers at SNP sites in linkage disequilibrium would likely indicate the same phenotype.

In certain embodiments, the SNP markers grouped within the same Sweep in Table 1 are all in linkage disequilibrium. In certain embodiments, the genetic marker is SNP A2. In certain embodiments, the genetic marker is any marker in Table 1 that is in linkage disequilibrium with SNP A2, or is a marker that is in strong linkage disequilibrium with SNP A2. In certain embodiments, the genetic marker is SNP B13. In certain embodiments, the genetic marker is any marker in Table 1 that is in linkage disequilibrium with SNP B13, or is a marker that is in strong linkage disequilibrium with SNP B13. In certain embodiments, the genetic marker is SNP C1. In certain embodiments, the genetic marker is SNP D1. In certain embodiments, the genetic marker is any marker in Table 1 that is in linkage disequilibrium with SNP D1, or is a marker that is in strong linkage disequilibrium with SNP D1. In certain embodiments, the genetic marker is SNP E10. In certain embodiments, the genetic marker is any marker in Table 1 that is in linkage disequilibrium with SNP E10, or is a marker that is in strong linkage disequilibrium with SNP E10.

Linkage disequilibrium can be assessed by suitable parameters known in the art, for example, in D value and r² (see details in, Hartl D L, Clark A G Principles of Population Genetics, Fourth Edition. Sinauer Associates, Inc). Both D value and r² represent the statistical correlation between two sites, and measure the strength of association between the two sites. In certain embodiments, an r² value of no less than 0.8 indicates a strong association (and therefore strong linkage disequilibrium) between two sites.

In certain embodiments, the SNP markers in strong linkage disequilibrium with SNP A2 include, SNP A1, SNP A3, SNP A4, SNP A5, SNP A6, SNP A7, SNP A8, SNP A9, SNP A10, SNP A11, SNP A12 and SNP A13.

In certain embodiments, the SNP markers in strong linkage disequilibrium with SNP B13 include, SNP B1, SNP B2, SNP B3, SNP B4, SNP B5, SNP B6, SNP B7, SNP B8, SNP B9, SNP B10, SNP B11, SNP B12, SNP B14, SNP B15, SNP B16, SNP B17, SNP B18, SNP B19, SNP B20, SNP B21, SNP B22, SNP B23, SNP B24, SNP B25, SNP B26, SNP B27, SNP B28, SNP B29, SNP B30, SNP B31, SNP B32, SNP B33, SNP B34, SNP B35, SNP B36, SNP B37, SNP B38, SNP B39, SNP B40, SNP B41, SNP B42, SNP B43, SNP B44, SNP B45, SNP B46, SNP B47, SNP B48, SNP B49, SNP B50, SNP B51, SNP B52, SNP B53, SNP B54, SNP B55, SNP B56, SNP B57, SNP B58, SNP B59, SNP B60, SNP B61, SNP B62, SNP B63, SNP B64, SNP B65, SNP B66, SNP B67, SNP B68, SNP B69, SNP B70, SNP B71, SNP B72, SNP B73, SNP B74, SNP B75, SNP B76, SNP B77, SNP B78, SNP B79, SNP B80, SNP B81, SNP B82, SNP B83, SNP B84, SNP B85, SNP B86, SNP B87, SNP B88, SNP B89, SNP B90 and SNP B91.

In certain embodiments, the SNP markers in strong linkage disequilibrium with SNP D1 include, SNP D2 and SNP D3.

In certain embodiments, the SNP markers in strong linkage disequilibrium with SNP E10 include, SNP E1, SNP E2, SNP E3, SNP E4, SNP E5, SNP E6, SNP E7, SNP E8, SNP E9, SNP E11, SNP E12, SNP E13, SNP E14, SNP E15, SNP E16, SNP E17, SNP E18, SNP E19, SNP E20, SNP E21, SNP E22 and SNP E23.

Pig is a diploid organism, and has two copies of chromosomes in its somatic cells. For a given SNP site on the chromosome, it is possible that the E allele SNP marker is present at the SNP site in both copies of the chromosomes (i.e. having dual copies of the E allele SNP marker at the SNP site), which suggests a E/E homozygous genotype; alternatively, it is also possible that the E allele SNP marker is present in only one of the two copies of the chromosomes (i.e. having a single copy of the E allele SNP marker at the SNP site), and O allele SNP marker is present on the other copy of the chromosome, which indicates a E/O heterozygous genotype; and it is further possible that the E allele SNP marker is absent in both copies of the chromosomes (i.e. having no copy of the E allele SNP marker at the SNP site), and O allele SNP marker is present on the both copies, which indicates a O/O homozygous genotype.

In certain embodiments, the presence of E/E genotype and/or E/O genotype at one of the SNP sites set forth in Sweep A in Table 1 indicates a phenotype of increased litter size.

In certain embodiments, the presence of E/E genotype at one of the SNP sites set forth in Sweep B in Table 1 indicates a phenotype of increased litter size.

In certain embodiments, the presence of E/E genotype and/or E/O genotype at the SNP site set forth in Sweep C in Table 1 indicates a phenotype of increased litter size.

In certain embodiments, the presence of E/O genotype at one of the SNP sites set forth in Sweep D in Table 1 indicates a phenotype of increased litter size.

In certain embodiments, the presence of E/E genotype and/or E/O genotype at one of the SNP sites set forth in Sweep E in Table 1 indicates a phenotype of increased litter size.

For germ cells containing only one copy of the chromosome, the presence of the E allele SNP marker at the SNP site indicates a phenotype of increased litter size.

Methods for Identifying a Pig with an Increased Litter Size

Another aspect of the present disclosure relates to methods of identifying a pig as producing an increased litter size. In certain embodiments, the methods comprise detecting the presence or absence of E allele SNP marker at one of the SNP sites within the genomic region selected from the group consisting of Sweep A, Sweep B, Sweep C, Sweep D, and Sweep E, and the presence of the E allele SNP marker indicates an increased litter size of the pig.

In certain embodiments, the Sweep A genomic region spans from Chr6: 122097788 to Chr6: 122217096 (NCBI build Sscrofa 10.2). In certain embodiments, the at least one SNP site within Sweep A genomic region is selected from the SNPs: A1-A13 as listed in Table 1. In certain embodiments, the at least one SNP site within Sweep A genomic region comprises SNP A2, or is in linkage disequilibrium with SNP A2. In certain embodiments, the presence of E/E genotype and/or E/O genotype at the SNP site in Sweep A indicates a phenotype of increased litter size.

In certain embodiments, the Sweep B genomic region spans from Chr6: 89403626 to Chr6: 90311616 (NCBI build Sscrofa 10.2). In certain embodiments, the at least one SNP site within Sweep B genomic region is selected from the SNPs: B1-B91 as listed in Table 1. In certain embodiments, the at least one SNP site within Sweep B genomic region comprises SNP B13, or is in linkage disequilibrium with SNP B13. In certain embodiments, the presence of E/E genotype at the SNP site in Sweep B indicates a phenotype of increased litter size.

In certain embodiments, the Sweep C genomic region locates at Chr7: 63714553 (NCBI build Sscrofa 10.2). In certain embodiments, the SNP site within Sweep C genomic region is SNP C1 as listed in Table 1. In certain embodiments, the presence of E/E genotype and/or E/O genotype at the SNP site in Sweep C indicates a phenotype of increased litter size.

In certain embodiments, the Sweep D genomic region spans from Chr15: 51799437 to Chr15: 51800356 (NCBI build Sscrofa 10.2). In certain embodiments, the at least one SNP site within Sweep D genomic region is selected from the SNPs: D1-D3 as listed in Table 1. In certain embodiments, the at least one SNP site within Sweep D genomic region comprises SNP D1, or is in linkage disequilibrium with SNP D1. In certain embodiments, the presence of E/O genotype at the SNP site in Sweep D indicates a phenotype of increased litter size.

In certain embodiments, the Sweep E genomic region spans from Chr3: 72655441 to Chr3: 72795872 (NCBI build Sscrofa 10.2). In certain embodiments, the at least one SNP site within Sweep E genomic region is selected from the SNPs: E1-E23 as listed in Table 1. In certain embodiments, the at least one SNP site within Sweep E genomic region comprises SNP E10, or is in linkage disequilibrium with SNP E10. In certain embodiments, the presence of E/E genotype and/or E/O genotype at the SNP site in Sweep E indicates a phenotype of increased litter size.

In certain embodiments, the E allele SNP marker is the high-frequency SNP marker at the corresponding SNP site in Erhualian pig genomes. In certain embodiments, the E allele SNP marker refers to the nucleotide shown under the column “E” of Table 1, at the corresponding SNP site. In certain embodiments, the O allele SNP marker refers to the nucleotide shown under the column “O” of Table 1, at the corresponding SNP site.

Any pig can be tested using the methods provided herein, including without limitation, a boar, a sow, and a piglet. The pigs can be of any suitable breed, for example, without limitation, Aksai Black Pied, American Landrace, American Yorkshire, Angeln Saddleback, Appalachian English, Arapawa Island, Auckland Island Pig, Australian Yorkshire, Babi Kampung, Ba Xuyen, Bantu, Bantu, Basque, Bazna, Beijing Black, Belarus Black Pied, Belgian Landrace, Bengali Brown Shannaj, Bentheim Black Pied, Berkshire, Bisaro, Black Slavonian, Black Canarian Pig, Breitovo, British Landrace, British Lop, British Saddleback, Bulgarian White, Cantonese, Chato Murciano, Chester White, Choctaw Hog, Creole Pig, Creole Pig, Cumberland Pig, Czech Improved White, Danish Landrace, Danish Protest Pig, Dermantsi Pied, Duroc, Dutch Landrace pig, East Balkan pig, Essex, Estonian Bacon, Fengjing pig, Finnish Landrance, Forest Mountain, French Landrace, Gascon, German Landrace, Gloucestershire Old Spot, Grice, Guinea Hog, Hampshire, Hante, Hereford, Hezuo, Iberian, Italian Landrace, Japanese Landrace, Jeju Black Pig, Jinhua, Kakhetian, Kele, Kemerovo, Korean Native Pig, Krskopolje, Kunekune, Lamcombe, Large Black, Large Black-white, Large White, Latvian White, Leicoma, Lithuanian Native, Lithuanian White, Lincolnshire Curly-Coated Pig, Livny, Malhado de Alcobaca, Mangalitsa, Meishan, Middle White, Minzhu, Middle White, Minzhu, Minokawa Buta, Mong Cai, Mora Romagnola, Moura, Mukota, Mulefoot, Murom, Myrhorod, Neijiang, Ningxiang, North Caucasian, North Siberian, Norwegian Landrace, Norwegian Yorkshire, Ossabaw Island, Oxford Sandy and Black, Philippine Native, Piétrain, Poland China, Red Wattle, Semirechye, Siberian Black Pied, Small Black, Small White, Spots, Surabaya Babi, Swabian-Hall, Swedish Landrace, Taihu pig, Tamworth, Thuoc Nhieu, Tibetan, Tokyo-X, Tsivilsk, Turopolje, Ukrainian Spotted Steppe, Ukrainian White Steppe, Urzhum, Vietnamese Potbelly, Welsh, Wessex Saddleback, West French White, Windsnyer, Wuzishan, Yanan, Yorkshire Blue and White. In certain embodiments, the pig is an Erhualian pig, a Taihu pig, a Sutai pig, a Meishan pig, a Fengjing pig, a Jiaxing Black pig. In certain embodiments, the pig is an offspring of Taihu pig. For example, the pig can be an offspring of Taihu pig crossed with any other pig breeds as listed above, or a pure bred of Taihu pig. In certain embodiments, the pig is an offspring of Erhualian pig, for example, an offspring of Erhualian pig crossed with any other pig breeds as listed above, or a pure bred of Erhualian pig.

A sample containing a nucleic acid molecule carrying the SNP site to be detected can be obtained from the pig. The sample can be a tissue sample, a somatic cell, a germ cell (such as a sperm cell and an egg cell), a cell extract, a cell nucleus extract, a genomic DNA sample, or a fragment of the genomic DNA sample. Optionally, the sample may be treated to release the nucleic acid, for example, by cell lysis, protease digestion, centrifugation, and DNA purification, etc. Such techniques are well-known in the art and can be adopted by people skilled in the art as appropriate. In certain embodiments, the sample contains genomic DNA of a pig, or at least a fragment of the genomic DNA which contains the SNP sites.

The sample can be used to detect the presence or absence of SNP markers disclosed herein. Any suitable methods can be used to conduct the detection described herein. The sample can be detected using methods known in the art, including for example, sequencing methods, PCR based methods, hybridization based methods, restriction digestion based methods, gel electrophoresis based methods and binding affinity methods.

In certain embodiments, the detecting comprises sequencing at least a fragment containing the SNP site in a nucleic acid sample from the pig. For example, a nucleic acid can be isolated from the pig and sequenced at least for the portion that contains the SNP site. Any suitable sequencing methods known in the art can be used.

In certain embodiments, the detecting comprises detecting an amplification product of at least a fragment containing the SNP site in a nucleic acid sample from the pig. The nucleic acid sample can be amplified using methods known in the art, for example, by PCR reaction in the presence of a pair of primers that flank the portion containing the SNP site. Briefly, the nucleic acid sample from the pig can be used as a template, and then amplified in the presence of a suitable polymerase, buffer, and temperature cycles, each of which includes a template denaturation step, a primer annealing step and a primer extension step, etc. The portion flanked by the primers can be amplified exponentially. The presence or absence and the size (or molecular weight) of the amplified product can be detected using, such as, gel electrophoresis. In certain embodiments, the presence or absence of the E allele SNP marker is revealed by presence (or absence) of the PCR amplification product. Optionally, the amplified product may be further sequenced to reveal the identity of the nucleotide at the SNP site, so that the presence or absence of the E allele SNP marker can be identified through the specific sequence at the SNP site.

In certain embodiments, the detecting comprises detecting hybridization of a probe to at least a fragment containing the SNP site in a nucleic acid sample from the pig. “Hybridization” as used herein refers to the process of hydrogen bond formation between a primer and a template DNA through base pairing, for example, between A and T pair, or between G and C pair. A probe can be an oligonucleotide molecule, which comprises natural nucleotides or non-natural nucleotides. The probe can optionally be labeled with a detectable moiety such as a radioligand, a fluorescent molecule or the like. The sample can be contacted with the probe and allow hybridization occur. The hybridization can be detected by methods known in the art, for example, by detecting the presence or absence of double-stranded hybridization product using a double-strand DNA dye, or by detecting the labeled probe bound to the target sequence immobilized on a chip, or by detecting the melting temperature (Tm) of the hybridization product, i.e. the temperature at which half of the hybridized product dissociates into single strands, or by detecting the fluorescent signal generated as a result of hybridization. In certain embodiments, the probe is designed to discriminating for different SNP markers at the SNP site, for example, specifically hybridize to the E allele SNP marker but not other markers, or vice versa, or alternatively, hybridizes to the E allele SNP marker at a Tm value detectably different from the Tm of other marker. By detecting the hybridization of the probe to the nucleic acid in the sample, people in the art can know the presence or absence of the E allele SNP marker at the interested SNP site.

In certain embodiments, the detecting comprises detecting a primer extension product of at least a fragment containing the SNP site in a nucleic acid sample from the pig. Primer extension can be performed with DNA polymerase and dideoxynucleoitde (ddNTP), which can be added to the 3′ end of the primer but prevents further extension as it does not have 3′-hydoxyl. To detect a SNP marker, primers can be designed to hybridize immediately upstream of the SNP site, so that the next nucleotide extended on the primer would be complementary to the SNP marker on the template. Such newly incorporated nucleotide can be analyzed for its mass (e.g. by MALDI-TOF Mass spectrometry), or by its unique fluorescence label. Alternatively, the 3′ end of primers can be designed to hybridize just to a selected SNP marker (e.g. the E allele SNP marker), if the selected SNP marker is present, then the 3′ end of the primer would complement to the template and allow primer extension to incorporate a labeled nucleotide, but in the absence of the selected SNP marker, the 3′ end of the primer would be in mismatch and thus would be allow primer extension.

In certain embodiments, the detecting comprises detecting restriction digestion product of at least a fragment containing the SNP site in a nucleic acid sample from the pig. In certain SNP sites, a SNP marker together with its adjacent nucleotides form a restriction site which can be specifically recognized and leaved by a restriction enzyme. Therefore, by treating the nucleic acid sample with such a restriction enzyme, the restriction sites on the nucleic acid sample, including the one containing the SNP site, would be cut to yield fragments with an expected pattern of sizes. However, if the SNP site harbors a different SNP marker which does not form such a restriction site, then the digestion of the nucleic acid sample by the restriction enzyme would result in at least one fragment having a size larger than expected.

In certain embodiments, the detecting comprises detecting gel electrophoresis results of at least a fragment containing the SNP site in a nucleic acid sample from the pig. In certain embodiments, the detection is based on temperature gradient gel electrophoresis. Briefly, mixture of the nucleic acid sample and a control nucleic acid are denatured and annealed. The nucleic acid sample and the control nucleic acid are identical expect that the sample contains an unknown SNP marker at the SNP site, whereas the control contains a known SNP marker (e.g. the E allele SNP marker) at the SNP site. If the SNP marker in the sample is the same as that in the control, then there will be one anneal product which show one band in electrophoresis. Otherwise, there would be four different anneal products (control, sample, hybrid of positive strand of the control and the negative strand of the sample, and a hybrid of negative strand of the control and the positive strand of the sample), which show detectably different bands in electrophoresis. In this way, the presence or absence of an interested SNP marker (e.g. E allele SNP marker) can be detected accordingly.

In certain embodiments, the detecting comprises detecting binding affinity of a protein to at least a fragment containing the SNP site in a nucleic acid sample from the pig. Certain DNA binding proteins (MutS protein from Thermus aquaticus) have been found to differentially bind to DNA molecules with different sequences, and thereby can be used to distinguish SNP markers.

In certain embodiments, more than one SNP site can be detected in one assay. For example, a microarray can be used to allow detection of multiple SNP markers at one time, in which multiple probes or primers for multiple SNP sites are immobilized at predetermined locations. By detecting the signal at each predetermined locations, the SNP markers at the multiple SNP sites can be determined in one assay.

In certain embodiments, a germ cell from a pig subject is tested in the methods. For example, a semen sample is collected from a male pig, and genomic DNA is obtained from the sample for further analysis and detection. If the E allele SNP marker is detected as present on at least one of the SNP site listed in Table 1, then the pig from which the semen sample is derived can be identified as producing an increased litter size. The identified male pig can be selected for further breeding, and the semen sample from the identified male pig can also be collected and used for further breeding, for example, through artificial fertilization or in vitro fertilization. Similarly, an egg sample can also be collected from a female pig, and the genomic DNA is obtained and tested. If the E allele SNP marker is detected as present on at least one of the SNP sites listed in Table 1, then the pig from which the egg sample is derived can be identified as producing an increased litter size. The identified female pig can be selected for further breeding, and the egg sample derived from the female pig may also be collected for further breeding, for example through artificial or in vitro fertilization.

In certain embodiments, the methods further comprise detecting for presence or absence of the E allele SNP marker in the SNP site of both copies of chromosomes. A sample derived from somatic cells can be used for such a detection. The presence or absence of the SNP sites on both copies of chromosomes can be detected simultaneously. For example, the detection signals are distinguishably different when both copies of the chromosomes contains the E allele SNP marker (i.e. E/E genotype), when only one copy of the chromosomes contains the E allele SNP marker (e.g. E/O genotype), or when neither copy contains the E allele SNP marker (e.g. O/O genotype). In certain embodiments, presence of the E allele SNP marker results in a detectable signal, which is not detected for O/O genotype, but detected for both E/O genotype and E/E genotype, with the E/E genotype having a signal with a higher magnitude. In certain embodiments, presence of the E allele SNP marker results in a signal of X (e.g. red color), the presence of the O allele SNP marker results in a signal of Y (e.g. green color), and the E/O genotype would result in a mixed signal of X and Y (e.g. both red and green color, which can be merged to show yellow color in the assay format).

In certain embodiments, with respect to SNP markers of Sweep A, the presence of a single copy or dual copies of the E allele SNP marker in the SNP site indicates an increased litter size of the pig. The genotypes of E/O and E/E at the SNP site of Sweep A both are correlated with a phenotype of increased litter size.

In certain embodiments, with respect to SNP markers of Sweep B, the presence of dual copies of the E allele SNP marker in the SNP site indicates an increased litter size of the pig. The genotype of E/E is correlated with a phenotype of increased litter size.

In certain embodiments, with respect to SNP markers of Sweep C, the presence of a single copy or dual copies of the E allele SNP marker in the SNP site indicates an increased litter size of the pig. The genotypes of E/O and E/E both are correlated with a phenotype of increased litter size.

In certain embodiments, with respect to SNP markers of Sweep D, the presence of a single copy of the E allele SNP marker in the SNP site indicates an increased litter size of the pig. The genotype of E/O is correlated with a phenotype of increased litter size.

In certain embodiments, with respect to SNP markers of Sweep E, the presence of a single copy or dual copies the E allele SNP marker in the SNP site indicates an increased litter size of the pig. The genotype of E/O and E/E both is correlated with a phenotype of increased litter size.

In certain embodiments, the methods further comprise selecting the pig for breeding if the pig is identified as producing an increased litter size. The pig as selected can be either a boar or a sow. In certain embodiments, a pig having dual or single copy number of the E allele marker at the corresponding SNP site produces at least 0.2, 0.3, 0.4. 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5 or more pigs per litter, when compared with a pig having no copy number of the E allele at the corresponding SNP site. In certain embodiments, a pig having dual or single copy number of the E allele marker at the corresponding SNP site produces at least 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% more pigs per litter, when compared with a pig having no copy number of the E allele at the corresponding SNP site.

Methods of Detecting the SNP Marker at a SNP Site within Sweep a Region

In another aspect, the present disclosure provides methods of detecting the SNP marker at a SNP site within Sweep A region in a pig. The methods comprise:

determining the presence or absence of an E allele SNP marker or an O allele SNP marker in at least one single nucleotide allele (SNP) site within Sweep A genomic region in a pig genome. In certain embodiments, the Sweep A genomic region spans from Chr6: 122097788 to Chr6: 122217096 (NCBI build Sscrofa 10.2). In certain embodiments, the at least one SNP site within Sweep A genomic region is selected from the SNPs: A1-A13 as listed in Table 1. In certain embodiments, the at least one SNP site within Sweep A genomic region comprises SNP A2, or is in linkage disequilibrium with SNP A2.

The E allele SNP marker is the high-frequency SNP marker at the corresponding SNP site in Erhualian pig genomes, and the E allele SNP markers are as shown in Table 1 under column “E”. The presence of the E allele SNP marker at the SNP site is indicative of an increased litter size of the pig. In certain embodiments, absence of the E allele SNP marker (i.e. no copy number of the E allele SNP marker) at the SNP site may suggest an absence of a phenotype of an increased litter size. In certain embodiments, the pigs identified as having E allele SNP marker in at least one SNP site in one or both copies of the chromosomes (i.e. having a single copy or dual copies of the E allele SNP marker at the SNP site) are identified as producing an increased litter size, and optionally, are selected for the breeding.

The O allele SNP marker is the high-frequency SNP marker at the corresponding SNP site in non-Erhualian pig genomes, and the O allele SNP markers are as shown in Table 1 under column “O”. In certain embodiments, the presence of the O allele SNP marker at the SNP site, for example in both copies of the chromosomes (i.e. dual copies of O allele SNP markers at the SNP site), can suggest lack of a phenotype of an increased litter size. In certain embodiments, the pigs identified as having O allele SNP marker at the SNP site in both copies of the chromosomes (i.e. dual copies of O allele SNP markers at the SNP site) are identified as not producing an increased litter size, and optionally, are excluded for the breeding.

In certain embodiments, the method further comprise determining the presence or absence of an E allele SNP marker or an O allele SNP marker at the SNP site of both copies of chromosomes, in which the SNP site is selected from SNP A1-A13 as shown in Table 1. The presence of an E allele SNP marker at both copies of the chromosomes (i.e. an E/E genotype), or at one of the copies of the chromosomes (i.e. an E/O genotype) is indicative of a phenotype of increased litter size. The presence of an O allele SNP marker at both copies of the chromosomes (i.e. an O/O genotype) is indicative of lack of a phenotype of increased litter size.

Methods of Detecting the SNP Marker at a SNP Site within Sweep B Region

In another aspect, the present disclosure provides methods of detecting the SNP marker at a SNP site within Sweep B region in a pig. The methods comprise: determining the presence or absence of an E allele SNP marker or an O allele SNP marker in at least one single nucleotide allele (SNP) site within Sweep B genomic region in a pig genome. In certain embodiments, the Sweep B genomic region spans from Chr6: 89403626 to Chr6: 90311616 (NCBI build Sscrofa 10.2). In certain embodiments, the at least one SNP site within Sweep B genomic region is selected from the SNPs: B1-B91 as listed in Table 1. In certain embodiments, the at least one SNP site within Sweep B genomic region comprises SNP B13, or is in linkage disequilibrium with SNP B13.

In certain embodiments, the method further comprise determining the presence or absence of an E allele SNP marker or an O allele SNP marker at the SNP site of both copies of chromosomes, in which the SNP site is selected from SNPs B1-B91 as shown in Table 1. The presence of an E allele SNP marker at both copies of the chromosomes (i.e. an E/E genotype) is indicative of a phenotype of increased litter size. The presence of an O allele SNP marker at both copies of the chromosomes (i.e. an O/O genotype), or at a single copy of the chromosomes (i.e. an E/O genotype) is indicative of lack of a phenotype of increased litter size.

Methods of Detecting the SNP Marker at a SNP Site within Sweep C Region

In another aspect, the present disclosure provides methods of detecting the SNP marker at a SNP site within Sweep C region in a pig. The methods comprise: determining the presence or absence of an E allele SNP marker or an O allele SNP marker in the single nucleotide allele (SNP) site within Sweep C genomic region in a pig genome. In certain embodiments, the Sweep C genomic region locates at Chr7: 63714553 (NCBI build Sscrofa 10.2). In certain embodiments, the SNP site within Sweep C genomic region is selected from the SNP: C1 as listed in Table 1.

In certain embodiments, the method further comprise determining the presence or absence of an E allele SNP marker or an O allele SNP marker at the SNP site of both copies of chromosomes, in which the SNP site is SNP C1 as shown in Table 1. The presence of an E allele SNP marker at both copies of the chromosomes (i.e. an E/E genotype), or at one of the copies of the chromosomes (i.e. an E/O genotype) is indicative of a phenotype of increased litter size. The presence of an O allele SNP marker at both copies of the chromosomes (i.e. an O/O genotype) is indicative of lack of a phenotype of increased litter size.

Methods of Detecting the SNP Marker at a SNP Site within Sweep D Region

In another aspect, the present disclosure provides methods of detecting the SNP marker at a SNP site within Sweep D region in a pig. The methods comprise: determining the presence or absence of an E allele SNP marker or an O allele SNP marker in at least one single nucleotide allele (SNP) site within Sweep D genomic region in a pig genome. In certain embodiments, the Sweep D genomic region spans from Chr15: 51799437 to Chr15: 51800356 (NCBI build Sscrofa 10.2). In certain embodiments, the at least one SNP site within Sweep D genomic region is selected from the SNPs: D1-D3 as listed in Table 1. In certain embodiments, the at least one SNP site within Sweep D genomic region comprises SNP D1, or is in linkage disequilibrium with SNP D1.

In certain embodiments, the method further comprise determining the presence or absence of an E allele SNP marker or an O allele SNP marker at the SNP site of both copies of chromosomes, in which the SNP site is selected from SNP D1-D3 as shown in Table 1. The presence of an E allele SNP marker at a single copy of the chromosomes (i.e. an E/O genotype) is indicative of a phenotype of increased litter size. The presence of an O allele SNP marker at both copies of the chromosomes (i.e. an O/O genotype), or at none of the chromosomes (i.e. an E/E genotype) is indicative of lack of a phenotype of increased litter size.

Methods of Detecting the SNP Marker at a SNP Site within Sweep E Region

In another aspect, the present disclosure provides methods of detecting the SNP marker at a SNP site within Sweep E region in a pig. The methods comprise: determining the presence or absence of an E allele SNP marker or an O allele SNP marker in at least one single nucleotide allele (SNP) site within Sweep E genomic region in a pig genome. In certain embodiments, the Sweep E genomic region spans from Chr3: 72655441 to Chr3: 72795872 (NCBI build Sscrofa 10.2). In certain embodiments, the at least one SNP site within Sweep E genomic region is selected from the SNPs: E1-E23 as listed in Table 1. In certain embodiments, the at least one SNP site within Sweep E genomic region comprises SNP E10, or is in linkage disequilibrium with SNP E10.

In certain embodiments, the method further comprises determining the presence or absence of an E allele SNP marker or an O allele SNP marker at the SNP site of both copies of chromosomes, in which the SNP site is selected from SNP E1-E23 as shown in Table 1. The presence of an E allele SNP marker at both copies of the chromosomes (i.e. an E/E genotype), or at one of the copies of the chromosomes (i.e. an E/O genotype) is indicative of a phenotype of increased litter size. Any methods described above can be used in the detection. The presence of an O allele SNP marker at both copies of the chromosomes (i.e. an O/O genotype) is indicative of lack of a phenotype of increased litter size.

The nucleotide at the SNP site can be determined either directly (e.g. by sequencing), or indirectly (e.g. by inferring from a known control sequence). Multiple SNP markers can be detected at one time.

Primers

In another aspect, the present disclosure provides isolated oligonucleotide primers, which are useful in distinguishing a nucleic acid sample carrying an E allele SNP marker from that carrying not.

In certain embodiments, the primers can be designed to amplify the target sequence regardless of the presence of the E allele SNP marker. As such, the amplified product can be further sequenced to determine the presence or absence of the E allele SNP marker at the SNP site. Such primers can be designed to flank the SNP site and be at least a few bases away from the SNP site. To provide a desirable PCR product, the amplicon can have a suitable size for example at least 100 bp, at least 200 bp, at least 500 bp, and the like.

In certain embodiments, the primers selectively amplify a polynucleotide fragment containing an E allele SNP marker in at least one of the SNP sites listed in Table 1. In such case, presence of the E allele SNP marker results in presence of amplification product which can be detected or further sequenced, while absence of the E allele SNP marker in the sample would result in absence of an amplified product. Such primers can be designed to have a 3′ end hybridizing exactly at the SNP site, and the 3′ end is complementary to the E allele SNP marker. Therefore, the presence of the E allele SNP marker at the SNP site would result in primer extension and production of the amplicon, while the absence of the E allele SNP marker would result in a mismatch at the 3′ end of the primer which would discourage formation of the amplicon.

In certain other embodiments, the primers selectively amplify a polynucleotide fragment lacking an E allele SNP marker in at least one of the SNP sites listed in Table 1. In certain embodiments, the primers selectively amplify a polynucleotide fragment containing an O allele SNP marker in at least one of the SNP sites listed in Table 1. In such case, presence of the E allele SNP marker results in absence of amplified product while absence of the E allele SNP marker (or, presence of the O allele SNP marker) results in presence of the amplified product. Similarly, the primers can be designed to have a 3′ end hybridizing right at the SNP site but complementary to the O allele SNP marker. Accordingly, presence of the O allele would result in amplification but not the E allele.

In certain embodiments, the primer is designed to have a 3′ end hybridizing immediately upstream of the SNP site. Such primer is useful in primer extension methods, in which only one nucleotide is incorporated to the 3′ end of the primer, and is complementary to the nucleotide at the SNP site (i.e. the SNP marker).

Probes

In another aspect, the present disclosure provides isolated oligonucleotide probes, which are useful in distinguishing a nucleic acid sample carrying an E allele SNP marker from that carrying not, based on hybridization with the sample.

In certain embodiments, the oligonucleotide probes are designed to selectively hybridize to a polynucleotide fragment containing an E allele SNP marker in one or more SNP sites listed in Table 1. For example, the probe binds to the E allele SNP marker at a different Tm value from that to the O allele SNP marker. As known in the art, one or more mismatches within a short oligonucleotide probe will decrease the Tm value of the DNA duplex formed thereby. Therefore, if an oligonucleotide probe hybridizes to a target sequence at the SNP site, the Tm value would vary according to the specific SNP marker that is presence at the SNP site, thereby indicating the identity of the SNP marker. The different Tm values can be determined using methods well known in the art.

In certain embodiments, the probes may be designed to have a fluorophore and a quencher conjugated at separate ends, such that in an intact probe, the fluorophore is quenched by the quencher. Such probe can be used in a TaqMan assay, in which the sample is amplified in the presence of the probe using PCR with Taq DNA polymerase. If the sample contains the E allele SNP marker, the probe will hybridize with the sample and consequently get degraded by the Taq DNA polymerase as it extends the DNA chain. This would result in separation of the flurophore and the quencher in the probe, thereby emitting fluorescence indicating presence of the E allele SNP marker. Similarly, the probes can also be designed to detect the presence of the O allele SNP marker in the similar way.

Kits

In another aspect, the present disclosure provides kits useful in the methods provided herein. The kits comprise the isolated oligonucleotide primers provided herein, or the isolated oligonucleotide probes provided herein. The kits may further comprise buffers, enzymes, or detecting label. The kits may further include instructions for use.

### EXAMPLES

### Example 1

Whole Genome Resequencing

With the availability of the domestic pig genome (Duroc pig), we performed a population whole genome resequencing for East Asian wild boars and five Chinese indigenous pig breeds, including Erhualian, Laiwu, Bama xiang, Diannan small-ear and Tibetan pigs. The chosen pig breeds represent two groups of populations with different ability in producing litter size. The Erhualian pigs, consisting of one breed of the Chinese Taihu local pigs, were chosen as the breed with larger litter size and compared to the other four Chinese local breeds and the wild boars. The pig populations were sequenced with a size of 11, 11, 9, 10 and 10 for Erhualian, Tibetan, Bama xiang, Laiwu, and Diannan small-ear pigs, and 10 for the wild boars, respectively. The aim was to identify important single nucleotide polymorphisms (SNPs) in the Erhualian genomes that can be potentially used in marker-assisted-selection (MAS) for improving the litter size trait in pigs.

Putative Selective Sweeps in Erhualian Pig Genomes

The domestic pigs in East Asia share a common origin from local wild boars [5,6]. Hence, the Erhualian pigs share a common ancestor (East Asian wild boars) with Laiwu, Bama xiang, Diannan small-ear and Tibetan pigs. The capability of producing a larger litter size for Erhualian pigs is a result of historic human-driven selection for an excess of economic benefit during the domestication and breeding process. Detecting the human-driven selection imprinting in the Erhualian genomes would facilitate the identification of genetic variations potentially responsible for increased litter size.

The genomic reads were aligned to the pig reference genome (NCBI build Sscrofa 10.2). The genomic variants were called using the SAM tools package. For a mutation that leads to an increased litter size in Erhualian pigs, it is possibly selected by human with a strong desire. Driven by this selection force, the causative mutation would reach at very high frequency in the Erhualian pigs, within quite a few generations. In the population genomes, an imprint of this kind of human-driven selection is a selective sweep spanning this favorable mutation. A selective sweep is referring to the reduction or elimination of variations in DNA sequences enclosing a beneficial mutation under strong positive natural selection or artificial selection. A selective sweep is caused by a lack of recombination in the genomic region that contains a favorable mutation. For flanking SNPs centered on the favorable mutation, a certain allele from each flanking SNP site would also increase in its allele frequency in the population, as a by-product of the selection against the favorable mutation, due to the lack of recombination between the flanking SNPs and the favorable mutation site. As a consequence, for a genomic region under a selective sweep, a decreased genetic diversity in the population of interest, and an increased differentiation level between the population and other populations are expected.

From the genomic differentiation, we developed a statistical method to detect the recent selective sweeps in the Erhualian pig genomes, by implementing Bayesian Markov Chain Monte Carlo to compare the posterior distributions of derived allele frequency between the Erhualian pigs and other pigs. The selective sweeps were intensively screened along the 18 autosomes in Erhualian pig genomes. Five putative selective sweeps were identified. The first putative selective sweep (Sweep A) is located on the pig chromosome 6, spanning through 122097788-122217096. A total of 13 SNP sites are identified in the sweep. The second putative selective sweep (Sweep B) is located on the pig chromosome 6, spanning through 89403626-90311616. A total of 91 SNP sites are identified in the sweep. The third putative selective sweep (Sweep C) is located on the pig chromosome 7, locating at Chr7: 63714553. One SNP site is identified in the sweep. The fourth putative selective sweep (Sweep D) is located on the pig chromosome 15, spanning through 51799437-51800356. A total of 3 SNP sites are identified in the sweep. The fifth putative selective sweep (Sweep E) is located on the pig chromosome 3, spanning through 72655441-72795872. A total of 23 SNP sites are identified in the sweep.

Co-Transmission of SNPs in a Sweep

Due to the scarcity of recombination, the flanking “hitchhiked” SNPs are historically co-selected with the favorable mutation at a causative SNP site within a selective sweep. Therefore, the flanking “hitchhiked” SNPs are always co-transmitted from parents to offspring together with the favorable mutation. Hence, in addition to the favorable mutation itself, the flanking “hitchhiked” SNPs are also useful in improving the litter size trait in a marker-assisted selection of breeding. Practically, it is not necessary to distinguish between the flanking “hitchhiked” SNPs and the causative SNP. The strong associations between flanking “hitchhiked” SNPs and the causative SNP would render a similar power in quantifying the effect in litter size regulation. One tag SNP can be randomly chosen to validate the effect in litter size regulation, as a representative of all SNPs organized in a selective sweep, irrespective of whether it is the causative or the “hitchhiked”.

If a tag SNP can function as an efficient molecular marker in improving litter size trait, the association between the tag SNP and other SNPs is an important indicator for the power that other SNPs have in facilitating the marker-assisted selection. A tightly associated SNP can be used in marker-assisted selection with a confidence as high as the tag SNP. The association between the tag SNP and other SNPs can be measured with a degree in linkage disequilibrium (LD), which reflects the occurrence of some combinations of alleles from two SNPs in a population more often than would be expected from a random formation from alleles based on their frequencies. A strong LD indicates one combination of alleles from two SNPs is always transmitted to offspring together.

In the total population, we consider a SNP X with two alleles, A and a, at frequencies P_{A} and pₐ, where P_{A}+pₐ=1. Similarly, a different SNP Y with alleles B and b has frequencies p_{B} and p_{b}, where p_{B}+p_{b}=1. If SNP X is in random association with SNP Y, the frequency of a gamete carrying any particular combination of alleles equals to the product of the frequencies of those alleles:

AB: P_{A}×p_{B}

ab: pₐ×pₕ

Ab: p_{A}×pₕ

aB: pₐ×p_{B}

SNPs in random association are said in a state of linkage equilibrium, and SNPs deviated from the random association are said in linkage disequilibrium. For example, under a condition that p_{AB}>P_{A}×p_{B}, it indicates that allele A on SNP X is transmitted together with allele B on SNP Y with a frequency higher than expected, where p_{AB} is the frequency of chromosomes carrying a combination of allele A on SNP X and allele B on SNP Y.

A test was widely used to verify whether two SNPs are in linkage disequilibrium. A linkage disequilibrium parameter, D [7], is given as:

D=p_{AB}*p_{ab}−p_{Ab}*p_{aB}

If SNP X and SNP Y are in strong linkage disequilibrium, a high D value is expected because we would observe an enrichment of non-recombinant gametes (AB and ab genotypes), and a deficiency of recombinant gametes (Aa and aB genotypes).

As a measure of linkage disequilibrium, the value of D has the limitation that it depends on the allele frequencies. Identical D values under different allele frequencies are indicative of different levels of linkage disequilibrium. A related but distinct parameter is r² [7]:

r²=D²/(p_{A}*p_{B}*pₐ*p_{b})

Here, r² is independent of the allelic frequencies at two SNPs of interests. The square root of r² is the correlation coefficient in allelic state between SNPs X and Y. A statistical significance test (Chi-squared test) can be performed on the r² parameter. The value of χ² is numerically equal to the r²*N, where N is the total number of chromosomes examined [7]. This χ² has one degree of freedom, and the associated probability can be calculated.

To validate the effect in litter size regulation, we randomly chose a tag SNP from each of the selective Sweeps A to E. The tag SNP for Sweep A was chr6:122,113,635 on chromosomes 6. The tag SNP for Sweep B was chr6:89,899,151 on chromosomes 6. The tag SNP for Sweep C was chr7: 63,714,553 on chromosomes 7. The tag SNP for Sweep D was chr15: 51,799,437 on chromosomes 15. The tag SNP for Sweep E was chr3: 72,759,645 on chromosome 3. The SNPs tightly associated with the tag SNPs are shown in Table 1, and the tag SNP is shown in bold. The associations between the tag SNP and the flanking SNPs are shown in Table 2.

Validating the Effect of the Sweep a in Litter Size Regulation in White Duroc Erhualian F2 Sows

192 F2 sows were selected from our previously developed White Duroc×Erhualian F2 sows pig population [3]. The F2 resource population was generated by crossing 2 White Duroc founder boars and 17 Erhualian founder sows as described in Ren et al [8]. QTLs for litter size were identified on chromosome 6, 7, 8 and 15 in the F2 resource population [3].

The genotypes at the tag SNP chr6:122,113,635 on the F2 sows are determined using the SNAPSHOT genotyping platform. The genotypes were successfully typed in 186 F2 sows. The numbers of individuals for the three genotypes C/C (i.e. the O/O genotype), T/C (i.e. the E/O genotype), and T/T (i.e. the E/E genotype) are 58, 85, and 43, respectively. The averaged litter size for F2 sows carrying the C/C genotype is 10.0676, with a standard deviation of 0.9348; the averaged litter size for F2 sows carrying the T/C genotype is 11.0982, with a standard deviation of 0.9206; and the averaged litter size for F2 sows carrying the T/T genotype is 11.8184, with a standard deviation of 0.9587.

A mixed procedure of SAS was employed to analyze the association between the tag SNP site and litter size using the following mixed model:

Yᵢⱼₖₗₘ=u+Aᵢ+Gⱼ+Bₖ+Pₗ+eᵢⱼₖₗₘ

Where: Yᵢⱼₖₗ is total or alive number of pig litter size; Aᵢ is additive effect (polygenic effect) of the ith individual; Gⱼ is the fixed effect of the jth SNP genotype; Bₖ is the fixed effect of the series of batches (k=1, 2, 3, 4); Pₗ is the random effect of the l^{th} parity (l=1, 2, 3), three parities are regarded as repeated measures.

The tag SNP chose from Sweep A was found in a statistically significant association with litter size (FIG. 1). On average, the F2 sows carrying the T/T genotype can produce 1.75 extra piglets per litter than F2 sows carrying the C/C genotype (p=0.0018); and the F2 sows carrying the T/C genotype can produce 1.03 extra piglets per litter than F2 sows carrying the C/C genotype (p=0.0225); a trend was observed that the F2 sows carrying the C/C genotype produce the smallest litter size.

Validating the Effects of Sweep a in Litter Size Regulation in Sutai Sows

We further examined the effect of the sweep A in a Sutai (Duroc×Meishan) sow population. A Sutai sow population of 192 individuals was chosen. The tag SNP chr6:122,113,635 was successfully typed in 185 sows of them.

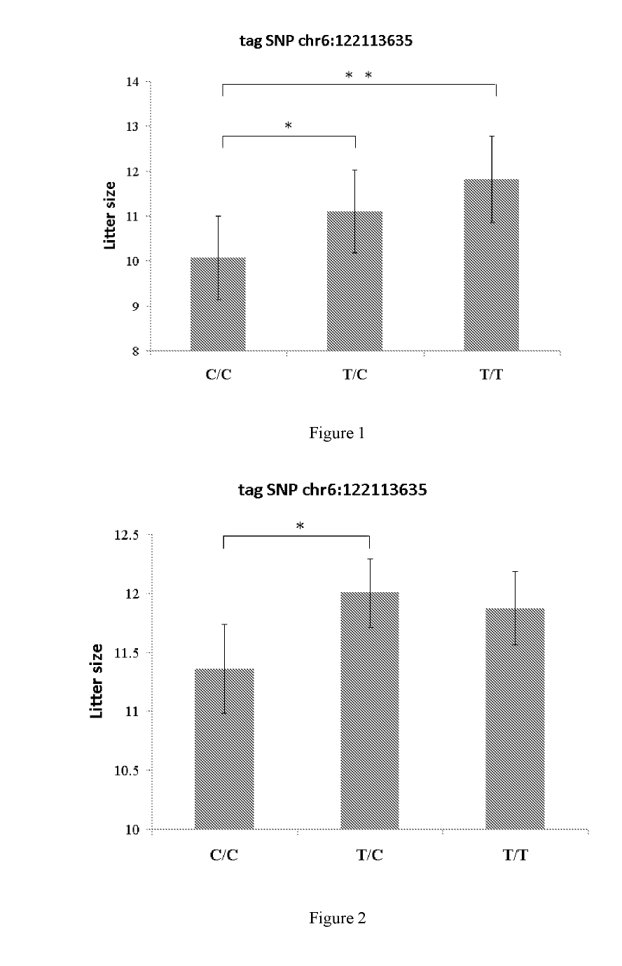

The numbers of individuals for the three genotypes C/C (i.e. the O/O genotype), T/C (i.e. the E/O genotype), and T/T (i.e. the E/E genotype) are 24, 103, and 58, respectively. As shown in FIG. 2, the averaged litter size for Sutai sows carrying the C/C genotype is 11.359, with a standard deviation of 0.3789; the averaged litter size for Sutai sows carrying the T/C genotype is 12.0029, with a standard deviation of 0.2912; and the averaged litter size for Sutai sows carrying the T/T genotype is 11.874, with a standard deviation of 0.3095.

A mixed procedure of SAS was employed to analyze the association between the tag SNP site and litter size using the following mixed model:

Yᵢⱼₖₗₘ=u+Aᵢ+Gⱼ+BₖPₗ+eᵢⱼₖₗₘ

Where: Yᵢⱼₖₗ is total or alive number of pig litter size; Aᵢ is additive effect (polygenic effect) of the ith individual; Gⱼ is the fixed effect of the jth SNP genotype; Bₖ is the fixed effect of the series of batches (k=1, 2, 3, 4); Pₗ is the random effect of the lth parity (l=1, 2, 3), three parities are regarded as repeated measures.

The association between the tag SNP and litter size trait was confirmed in the Sutai sow population. On average, the Sutai sows carrying the T/C genotype can produce 0.64 extra piglets per litter than Sutai sows carrying the C/C genotype (p=0.0251); though the difference is not statistically significant (p=0.0873) in Sutai sows carrying either the T/T genotype or the C/C genotype, the individuals carrying T/T genotype produce 0.51 extra piglets per litter than individuals carrying C/C genotype. A trend was observed that the Sutai sows carrying C/C genotype produce the smallest litter size, which is similar to that observed in White Duroc×Erhualian F2 sows.

Validating the Effect of the Sweep B in Litter Size Regulation in White Duroc×Erhualian F2 Sows

192 F2 sows were selected from our previously developed White Duroc×Erhualian F2 sows pig population [3]. The F2 resource population was generated by crossing 2 White Duroc founder boars and 17 Erhualian founder sows as described in Ren et al [8]. QTLs for litter size were identified on chromosome 6, 7, 8 and 15 in the F2 resource population [3].

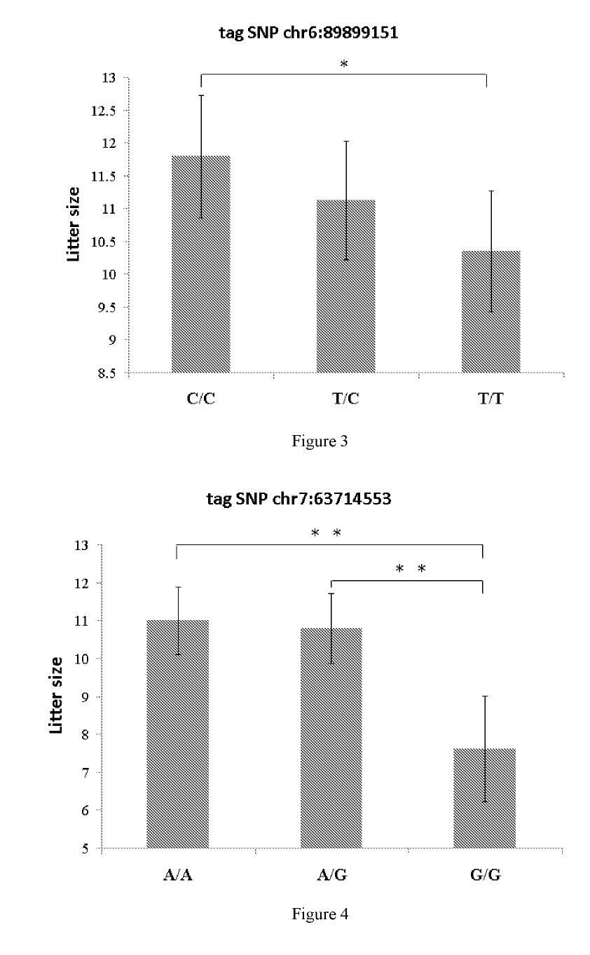

The genotypes at the tag SNP chr6: 89,899,151 on the 192 F2 sows are determined using the SNAPSHOT genotyping platform. The genotypes were successfully typed in 161 individuals of them. As shown in Table 1, at the tag SNP chr6: 89,899,151, the E allele SNP marker is C, and the O allele SNP marker is T. The numbers of individuals for the three genotypes C/C (i.e. E/E genotype), T/C (i.e. E/O genotype), and T/T (i.e. O/O genotype) are 43, 69 and 49 respectively. As shown in FIG. 3, the averaged litter size for F2 sows carrying the C/C genotype is 11.7932, with a standard deviation of 0.9316; the averaged litter size for F2 sows carrying the T/C genotype is 11.1217, with a standard deviation of 0.9001; and the averaged litter size for F2 sows carrying the T/T genotype is 10.3489, with a standard deviation of 0.9244.

A mixed procedure of SAS was employed to analyze the association between the tag SNP site and litter size using the following mixed model:

Yᵢⱼₖₗₘ=u+Aᵢ+Gⱼ+Bₖ+Pₗ+eᵢⱼₖₗₘ

Where: Yᵢⱼₖₗ is total or alive number of pig litter size; Aᵢ is additive effect (polygenic effect) of the ith individual; Gⱼ is the fixed effect of the jth SNP genotype; Bₖ is the fixed effect of the series of batches (k=1, 2, 3, 4); Pₗ is the random effect of the l^{th} parity (l=1, 2, 3), three parities are regarded as repeated measures.

The tag SNP was found in a statistically significant association with litter size trait. On average, the F2 sows carrying the C/C genotype can produce 1.44 extra piglets per litter than F2 sows carrying the T/T genotype (p=0.0127); though the statistical test render an insignificant result (p=0.1226) when comparing F2 sows carrying the T/C genotype and the T/T genotype, the individuals carrying T/C genotype produce 0.77 extra piglets per litter than individuals carrying T/T genotype. A trend was observed that the F2 sows carrying the T/T genotype have the smallest litter size.

Validating the Effect of the Sweep C in Litter Size Regulation in White Duroc Erhualian F2 Sows

192 F2 sows were selected from our previously developed White Duroc×Erhualian F2 sows pig population [3]. The F2 resource population was generated by crossing 2 White Duroc founder boars and 17 Erhualian founder sows as described in Ren et al [8]. QTLs for litter size were identified on chromosome 6, 7, 8 and 15 in the F2 resource population [3].

The genotypes at the tag SNP chr7: 63,714,553 on the 192 F2 sow are determined using the SNAPSHOT genotyping platform. The genotypes were successfully typed in 185 individuals of them. As shown in Table 1, at the tag SNP chr7: 63,714,553, the E allele SNP marker is A, and the O allele SNP marker is G. The numbers of individuals for the three genotypes G/G (i.e. O/O genotype), A/G (i.e. E/O genotype), and A/A (i.e. O/O genotype) are 6, 58, and 121, respectively. As shown in FIG. 4, the averaged litter size for F2 sows carrying the G/G genotype is 7.6198, with a standard deviation of 1.3942; the averaged litter size for F2 sows carrying the A/G genotype is 10.7951, with a standard deviation of 0.9178; and the averaged litter size for F2 sows carrying the A/A genotype is 10.9955, with a standard deviation of 0.8884.

A mixed procedure of SAS was employed to analyze the association between the tag SNP site and litter size using the following mixed model:

Yᵢⱼₖₗₘ=u+Aᵢ+Gⱼ+Bₖ+Pₗ+eᵢⱼₖₗₘ

Where: Yᵢⱼₖₗ is total or alive number of pig litter size; Aᵢ is additive effect (polygenic effect) of the ith individual; Gⱼ is the fixed effect of the jth SNP genotype; Bₖ is the fixed effect of the series of batches (k=1, 2, 3, 4); Pₗ is the random effect of the l^{th} parity (l=1, 2, 3), three parities are regarded as repeated measures.

The tag SNP was found in a statistically significant association with litter size trait. On average, the F2 sows carrying the A/G genotype can produce 3.17 extra piglets per litter than F2 sows carrying the G/G genotype (p=0.0086); and the statistical test render an significant result (p=0.0045) when comparing F2 sows carrying the G/G genotype and the A/A genotype, the individuals carrying A/A genotype produce 3.37 extra piglets per litter than individuals carrying G/G genotype. A trend was observed that the F2 sows carrying the G/G genotype have the smallest litter size.

Validating the Effects of Sweep C in Litter Size Regulation in Sutai Sows

We further examined the effect of the sweep C in a Sutai (Duroc×Meishan) sow population. A Sutai sow population of 192 individuals was chosen. The tag SNP chr7: 63,714,553 was successfully typed in 181 sows of them.

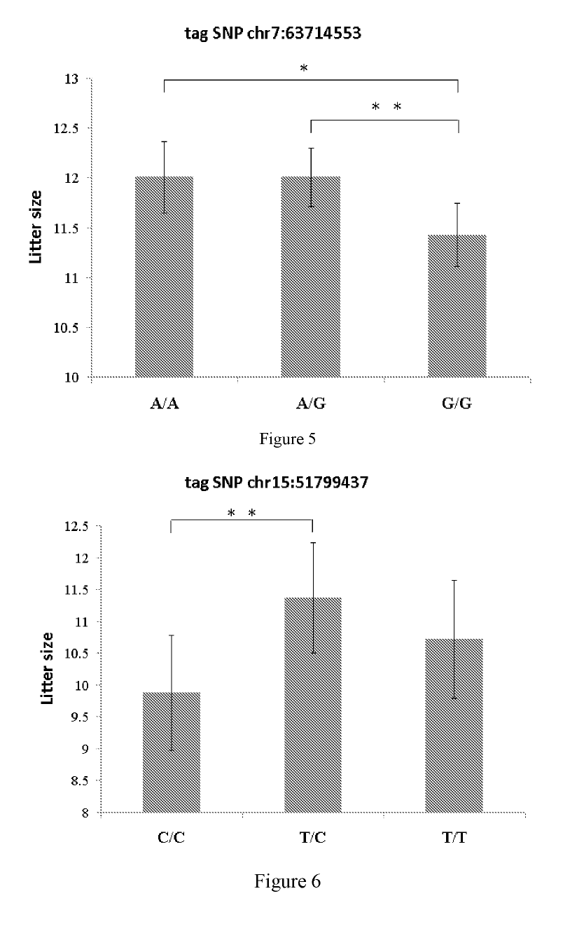

The numbers of individuals for the three genotypes G/G (i.e. the O/O genotype), G/A (i.e. the E/O genotype), and A/A (i.e. the E/E genotype) are 53, 97, and 31, respectively. As shown in FIG. 5, the averaged litter size for Sutai sows carrying the A/A genotype is 12.0069, with a standard deviation of 0.3598; the averaged litter size for Sutai sows carrying the A/G genotype is 12.0076, with a standard deviation of 0.2905; and the averaged litter size for Sutai sows carrying the G/G genotype is 11.4269, with a standard deviation of 0.3199.

A mixed procedure of SAS was employed to analyze the association between the tag SNP site and litter size using the following mixed model:

Yᵢⱼₖₗₘ=u+Aᵢ+Gⱼ+Bₖ+Pₗ+eᵢⱼₖₗₘ

Where: Yᵢⱼₖₗ is total or alive number of pig litter size; Aᵢ is additive effect (polygenic effect) of the ith individual; Gⱼ is the fixed effect of the jth SNP genotype; Bₖ is the fixed effect of the series of batches (k=1, 2, 3, 4); Pₗ is the random effect of the lth parity (1=1, 2, 3), three parities are regarded as repeated measures.

The association between the tag SNP chr7: 63,714,553 and litter size trait was confirmed in the Sutai sow population. On average, the Sutai sows carrying the A/G genotype can produce 0.58 extra piglets per litter than Sutai sows carrying the G/G genotype (p=0.0034); and the statistical test render an significant result (p=0.0384) when comparing F2 sows carrying the A/A genotype and the G/G genotype, the individuals carrying A/A genotype produce 0.57 extra piglets per litter than individuals carrying G/G genotype. A trend was observed that the Sutai sows carrying G/G genotype produce the smallest litter size, which is similar to that observed in White Duroc×Erhualian F2 sows.

Validating the Effect of the Sweep D in Litter Size Regulation in White Duroc Erhualian F2 Sows

192 F2 sows were selected from our previously developed White Duroc×Erhualian F2 sows pig population [3]. The F2 resource population was generated by crossing 2 White Duroc founder boars and 17 Erhualian founder sows as described in Ren et al [8]. QTLs for litter size were identified on chromosome 6, 7, 8 and 15 in the F2 resource population [3].

The genotypes at the tag SNP chr15: 51,799,437 were successfully typed in 183 individuals of them. As shown in Table 1, at the tag SNP chr15: 51,799,437, the E allele SNP marker is T, and the O allele SNP marker is C. The numbers of individuals for the three genotypes C/C (i.e. O/O genotype), T/C (i.e. E/O genotype), and T/T (i.e. E/E genotype) are 53, 87, and 43, respectively. As shown in FIG. 6, the averaged litter size for F2 sows carrying the C/C genotype is 9.8743, with a standard deviation of 0.9046; the averaged litter size for F2 sows carrying the T/C genotype is 11.3673, with a standard deviation of 0.8657; and the averaged litter size for F2 sows carrying the T/T genotype is 10.7179, with a standard deviation of 0.9227.

A mixed procedure of SAS was employed to analyze the association between the tag SNP site and litter size using the following mixed model:

Yᵢⱼₖₗₘ=u+Aᵢ+Gⱼ+Bₖ+Pₗ+eᵢⱼₖₗₘ

Where: Yᵢⱼₖₗ is total or alive number of pig litter size; Aᵢ is additive effect (polygenic effect) of the ith individual; Gⱼ is the fixed effect of the jth SNP genotype; Bₖ is the fixed effect of the series of batches (k=1, 2, 3, 4); Pₗ is the random effect of the lth parity (l=1, 2, 3), three parities are regarded as repeated measures.

The tag SNP was found in a statistically significant association with litter size trait. On average, the F2 sows carrying the T/C genotype can produce 1.49 extra piglets per litter than F2 sows carrying the C/C genotype (p=0.0018); though the statistical test render an insignificant result (p=0.1211) when comparing F2 sows carrying the C/C genotype and the T/T genotype, the individuals carrying T/T genotype produce 0.84 extra piglets per litter than individuals carrying C/C genotype. A trend was observed that the F2 sows carrying the C/C genotype have the smallest litter size.

Validating the Effects of Sweep D in Litter Size Regulation in Sutai Sows

We further examined the effect of the sweep D in a Sutai (Duroc×Meishan) sow population. A Sutai sow population of 192 individuals was chosen. The tag SNP chr15:51,799,437 was successfully typed in 185 sows of them.

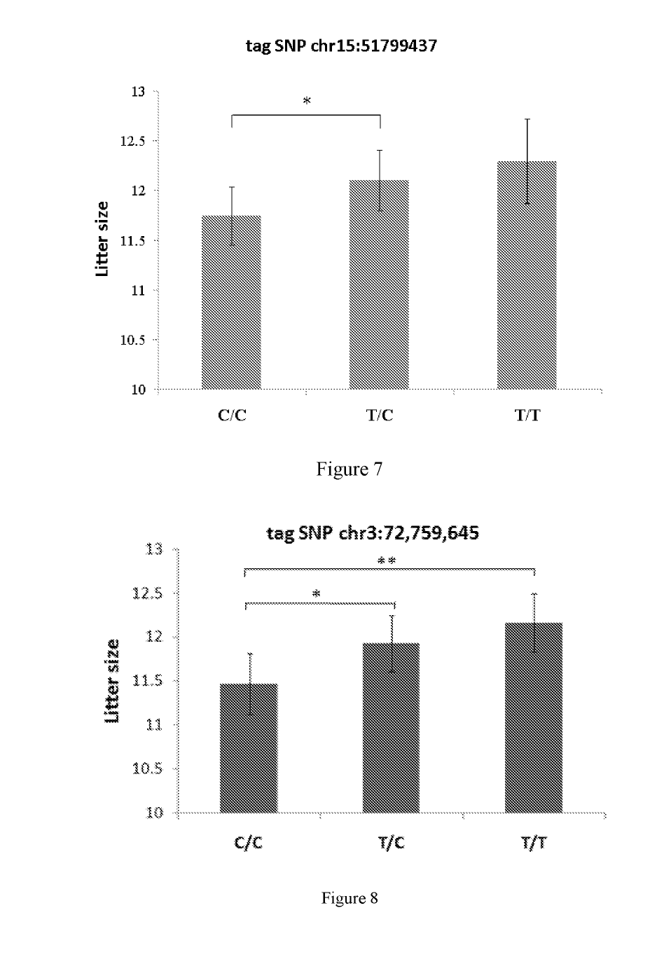

The numbers of individuals for the three genotypes C/C, T/C, and T/T are 84, 87, and 14, respectively. As shown in FIG. 7, the averaged litter size for Sutai sows carrying the C/C genotype is 11.742, with a standard deviation of 0.2919; the averaged litter size for Sutai sows carrying the T/C genotype is 12.0997, with a standard deviation of 0.3037; and the averaged litter size for Sutai sows carrying the T/T genotype is 12.2924, with a standard deviation of 0.4272.

A mixed procedure of SAS was employed to analyze the association between the tag SNP site and litter size using the following mixed model:

Yᵢⱼₖₗₘ=u+Aᵢ+Gⱼ+Bₖ+Pₗ+eᵢⱼₖₗₘ

Where: Yᵢⱼₖₗ is total or alive number of pig litter size; Aᵢ is additive effect (polygenic effect) of the ith individual; Gⱼ is the fixed effect of the jth SNP genotype; Bₖ is the fixed effect of the series of batches (k=1, 2, 3, 4); Pₗ is the random effect of the lth parity (l=1, 2, 3), three parities are regarded as repeated measures.

The association between the tag SNP chr15: 51,799,437 and litter size trait was confirmed in the Sutai sow population. On average, the Sutai sows carrying the T/C genotype can produce 0.36 extra piglets per litter than Sutai sows carrying the C/C genotype (p=0.0436); though the difference is not statistically significant (p=0.1153) in Sutai sows carrying either the T/T genotype or the C/C genotype, the individuals carrying T/T genotype produce 0.55 extra piglets per litter than individuals carrying C/C genotype. A trend was observed that the Sutai sows carrying C/C genotype produce the smallest litter size, which is similar to that observed in White Duroc×Erhualian F2 sows.

Validating the Effect of the Sweep E in Litter Size Regulation in Large White Sows

434 Large White sows were selected to validate the effect of the sweep E in litter size regulation. The genotypes at the tag SNP chr3: 72,759,645 were successfully typed in the sow population. The numbers of individuals for the three genotypes C/C (i.e. O/O genotype), T/C (i.e. E/O genotype), and T/T (i.e. E/E genotype) are 84, 218, and 132, respectively. As shown in FIG. 8, the averaged litter size for the Large White sows carrying the C/C genotype is 11.4712, with a standard deviation of 0.3469; the averaged litter size for the Large White sows carrying the T/C genotype is 11.9252, with a standard deviation of 0.3182; and the averaged litter size for the Large White sows carrying the T/T genotype is 12.1636, with a standard deviation of 0.325.

A mixed procedure of SAS was employed to analyze the association between the tag SNP site and litter size using the following mixed model:

Yᵢⱼₖₗₘ=u+Aᵢ+Gⱼ+Bₖ+Pₗ+eᵢⱼₖₗₘ

Where: Yᵢⱼₖₗ is total or alive number of pig litter size; Aᵢ is additive effect (polygenic effect) of the ith individual; Gⱼ is the fixed effect of the jth SNP genotype; Bₖ is the fixed effect of the series of batches (k=1, 2, 3, 4); Pₗ is the random effect of the lth parity (l=1, 2, 3), three parities are regarded as repeated measures.

The tag SNP was found in a statistically significant association with litter size trait. On average, the Large White sows carrying the T/T genotype can produce 0.69 extra piglets per litter than the Large White sows carrying the C/C genotype (p=0.0015); the Large White sows carrying the T/C genotype can produce 0.45 extra piglets per litter than the Large White sows carrying the C/C genotype (p=0.0221); A trend was observed that the Large White sows carrying the T/T genotype have the largest litter size.

Validating the Effect of the Sweep E in Litter Size Regulation in Large White×Landrace (LL) F1 Crossbred Sows

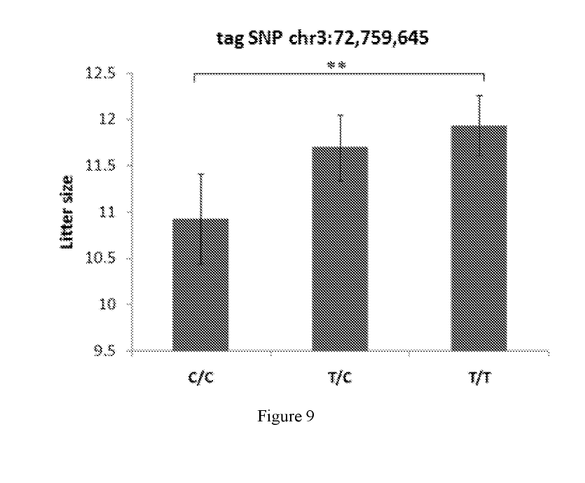

404 LL F1 sows were selected to validate the effect of the sweep E in litter size regulation. The genotypes at the tag SNP chr3: 72,759,645 were successfully typed in the LL F1 sow population. The numbers of individuals for the three genotypes C/C, T/C, and T/T are 84, 218, and 132, respectively. As shown in FIG. 9, the averaged litter size for the LL F1 sows carrying the C/C genotype is 10.9214, with a standard deviation of 0.4841; the averaged litter size for the LL F1 sows carrying the T/C genotype is 11.6953, with a standard deviation of 0.3551; and the averaged litter size for the LL F1 sows carrying the T/T genotype is 11.9334, with a standard deviation of 0.327.

A mixed procedure of SAS was employed to analyze the association between the tag SNP site and litter size using the following mixed model:

Yᵢⱼₖₗₘ=u+Aᵢ+Gⱼ+BₖPₗ+eᵢⱼₖₗₘ

Where: Yᵢⱼₖₗ is total or alive number of pig litter size; Aᵢ is additive effect (polygenic effect) of the ith individual; Gⱼ is the fixed effect of the jth SNP genotype; Bₖ is the fixed effect of the series of batches (k=1, 2, 3, 4); Pₗ is the random effect of the lth parity (l=1, 2, 3), three parities are regarded as repeated measures.

The tag SNP was found in a statistically significant association with litter size trait. On average, the LL F1 sows carrying the T/T genotype can produce 1.012 extra piglets per litter than the LL F1 sows carrying the C/C genotype (p=0.0015); the LL F1 sows carrying the T/C genotype can produce 0.7739 extra piglets per litter than the LL F1 sows carrying the C/C genotype (p=0.0558); A trend was observed that the LL F1 sows carrying the T/T genotype have the largest litter size.

### REFERENCE

1. Zhang Z (1986) Pig Breeds in China. Shanghai Scientific and Technical Publishers, Shanghai, China.
2. Rothschild M F, Messer L, Day A, Wales R, Short T, et al. (2000) Investigation of the retinol-binding protein 4 (RBP4) gene as a candidate gene for increased litter size in pigs. Mamm Genome 11: 75-77.
3. Li K, Ren J, Xing Y, Zhang Z, Ma J, et al. (2009) Quantitative trait loci for litter size and prenatal loss in a White Duroc×Chinese Erhualian resource population. Anim Genet 40: 963-966.
4. Wilkie P J, Paszek A A, Beattie C W, Alexander L J, Wheeler M B, et al. (1999) A genomic scan of porcine reproductive traits reveals possible quantitative trait loci (QTLs) for number of corpora lutea. Mamm Genome 10: 573-578.
5. Larson G, Dobney K, Albarella U, Fang M, Matisoo-Smith E, et al. (2005) Worldwide phylogeography of wild boar reveals multiple centers of pig domestication. Science 307: 1618-1621.
6. Wu G S, Yao Y G, Qu K X, Ding Z L, Li H, et al. (2007) Population phylogenomic analysis of mitochondrial DNA in wild boars and domestic pigs revealed multiple domestication events in East Asia. Genome Biol 8: R245.
7. Hartl D L, Clark A G Principles of Population Genetics, Fourth Edition. Sinauer

Associates, Inc
8. Ren D R, Ren J, Xing Y Y, Guo Y M, Wu Y B, et al. (2009) A genome scan for quantitative trait loci affecting male reproductive traits in a White Duroc×Chinese Erhualian resource population. J Anim Sci 87: 17-23.

TABLE 1
SNP markers on Chromosomes 6, 7, 15 and 3

| | | | | | | | | | | 20 bp flanking sequences |
|---|---|---|---|---|---|---|---|---|---|---|
| | | | | | | | | | | on each side, centered on |
| | | | | | | | | | | the SNP site (plus strand) |
| | | | | | | | | | Exon/ | (in which the SNP site is |
| | | Chromo- | | Alleles | | | Associated | Associated | Intron | illustrated by |
| Sweep | SNP | some | Position | E | O | Class | gene 1 | gene2 | ID | [E allele/O allele]) |
| A | A1 | 6 | 122097788 | A | C | Intronic | LPHN2 | — | 1 | AAAATACATTCTCAGAGTTC |
| | | | | | | | | | | [A/C] |
| | | | | | | | | | | CATTGTGGCGCAGCGGAAAC |
| A | A2 | 6 | 122113635 | T | C | Intergenic | LPHN2 | ELTD1 | — | ACCGACCACTAGTCGCCACC |
| | | | | | | | | | | [T/C] |
| | | | | | | | | | | TCTCTCTCCATTTCATTTCG |
| A | A3 | 6 | 122123140 | A | G | Intergenic | LPHN2 | ELTD1 | — | TAAAGATACAATATACTTTT |
| | | | | | | | | | | [A/G] |
| | | | | | | | | | | TCATCATTCAGAATAATGCA |
| A | A4 | 6 | 122125228 | G | T | Intergenic | LPHN2 | ELTD1 | — | TCACTAACTACAGGTGCTGA |
| | | | | | | | | | | [G/T] |
| | | | | | | | | | | AGGTGCTTTCGGTTGTTGTT |
| A | A5 | 6 | 122126620 | A | G | Intergenic | LPHN2 | ELTD1 | — | TTTCTCTGATGAGATTTTAA |
| | | | | | | | | | | [A/G] |
| | | | | | | | | | | TACCTACAGAGCACAGTCCT |
| A | A6 | 6 | 122126799 | T | A | Intergenic | LPHN2 | ELTD1 | — | AAATACAGACTTGAAGACCA |
| | | | | | | | | | | [T/A] |
| | | | | | | | | | | TTGAAGCAAATGCATACTTT |
| A | A7 | 6 | 122148636 | T | C | Intergenic | LPHN2 | ELTD1 | — | TCTCTCCACGCTGAGTAAAT |
| | | | | | | | | | | [T/C] |
| | | | | | | | | | | TTGGTCCAAGAAGGCCAAAC |
| A | A8 | 6 | 122178848 | T | C | Intergenic | LPHN2 | ELTD1 | — | CTGTGAGCTGTGGTGTGGGT |
| | | | | | | | | | | [T/C] |
| | | | | | | | | | | GCAGACGCGGCTCGGATCCC |
| A | A9 | 6 | 122178862 | A | G | Intergenic | LPHN2 | ELTD1 | — | GTGGGTCGCAGACGCGGCTC |
| | | | | | | | | | | [A/G] |
| | | | | | | | | | | GATCCCGTGTTGCTGTGGCT |
| A | A10 | 6 | 122178870 | C | T | Intergenic | LPHN2 | ELTD1 | — | CAGACGCGGCTCGGATCCCG |
| | | | | | | | | | | [C/T] |
| | | | | | | | | | | GTTGCTGTGGCTCTGGCGTA |
| A | A11 | 6 | 122197541 | G | A | Intergenic | LPHN2 | ELTD1 | — | AGCCGGGGGGGGGGGGAGGT |
| | | | | | | | | | | [G/A] |
| | | | | | | | | | | CTTTTCAAAGCACAGAGAAA |
| A | A12 | 6 | 122197588 | G | A | Intergenic | LPHN2 | ELTD1 | — | CCTAAAATTAAGAGTGATAA |
| | | | | | | | | | | [G/A] |
| | | | | | | | | | | CTAGAGGAACCTACAAAAAC |
| A | A13 | 6 | 122217096 | A | T | Intergenic | LPHN2 | ELTD1 | — | TTTGGGCCTGTTCTGTTGTT |
| | | | | | | | | | | [A/T] |
| | | | | | | | | | | AAATACATAACTGAAGGAGT |
| B | B1 | 6 | 89403626 | C | T | Intergenic | CAP1 | COL9A2 | — | TTTTAAGCAAGGGACTAACA |
| | | | | | | | | | | [C/T] |
| | | | | | | | | | | AATTGAATTTGTTTGGAGAG |
| B | B2 | 6 | 89412355 | G | A | Intergenic | CAP1 | COL9A2 | — | ATAGGATCCCCAGCATACTT |
| | | | | | | | | | | [G/A] |
| | | | | | | | | | | TCATCAGCATCCTCCTCCTC |
| B | B3 | 6 | 89412733 | G | A | Intergenic | CAP1 | COL9A2 | — | TGTGCCACAGCAGTGACCCA |
| | | | | | | | | | | [G/A] |
| | | | | | | | | | | GCAGCTGCAGTGACAATGCC |
| B | B4 | 6 | 89413181 | C | T | Intergenic | CAP1 | COL9A2 | — | TCAACGAGCTCAACTAGAAT |
| | | | | | | | | | | [C/T] |
| | | | | | | | | | | CATGAGGATGTGGGTTTGCT |
| B | B5 | 6 | 89413302 | G | A | Intergenic | CAP1 | COL9A2 | — | ATTGCAGTGGCTAAGGCGTC |
| | | | | | | | | | | [G/A] |
| | | | | | | | | | | GGCAGCTGCAGCTCCAATTC |
| B | B6 | 6 | 89413372 | C | A | Intergenic | CAP1 | COL9A2 | — | GCAGGTGTGGCCCTAAAATG |
| | | | | | | | | | | [C/A] |
| | | | | | | | | | | AAAAAAGAAAAAGAAAAGAA |
| B | B7 | 6 | 89413471 | G | T | Intergenic | CAP1 | COL9A2 | — | CATGTCAATAACTATTTTAT |
| | | | | | | | | | | [G/T] |
| | | | | | | | | | | TATTTATTTATTTATTTATT |
| B | B8 | 6 | 89413560 | T | C | Intergenic | CAP1 | COL9A2 | — | CCATATGCTGCAGAGGCAGC |
| | | | | | | | | | | [T/C] |
| | | | | | | | | | | GAAAAAGAAAAAAATTATAA |
| B | B9 | 6 | 89414704 | G | A | Intergenic | CAP1 | COL9A2 | — | GGTTCATTCTTGTAGAGGAA |
| | | | | | | | | | | [G/A] |
| | | | | | | | | | | TTACTATGGTAGGTGGAAAA |
| B | B10 | 6 | 89414733T | T | C | Intergenic | CAP1 | COL9A2 | — | GTAGGTGGAAAAACAAGAGC |
| | | | | | | | | | | [T/C] |
| | | | | | | | | | | GACATTTGAGTTTCTTTTTT |
| B | B11 | 6 | 89431138 | G | C | Intronic | COL9A2 | — | 24 | AGAGGGGCGTGGCTAGGAGC |
| | | | | | | | | | | [G/C] |
| | | | | | | | | | | GGGATAGGGGGAACCCGGTG |
| B | B12 | 6 | 89888614 | A | G | Intergenic | C18orf1 | CEP192 | — | GCCACAGCAACGCAGGATCC |
| | | | | | | | | | | [A/G] |
| | | | | | | | | | | AGCCAAGTCTGCAACCTACA |
| B | B13 | 6 | 89899151 | C | T | Intergenic | C18orf1 | CEP192 | — | CGTGAATGGAGATAGATTTT |
| | | | | | | | | | | [C/T] |
| | | | | | | | | | | GGTTTTAGAAAAACCCCAGA |
| B | B14 | 6 | 89902460 | G | A | Intergenic | C18orf1 | CEP192 | — | CTGGGAATTTTATATGCAGC |
| | | | | | | | | | | [G/A] |
| | | | | | | | | | | GGGCAGCCAAAAAAAAAAAA |
| B | B15 | 6 | 89906636 | T | C | Intergenic | C18orf1 | CEP192 | — | ATTGAAGTTTGTGGCAATCC |
| | | | | | | | | | | [T/C] |
| | | | | | | | | | | ATGTCAAACAAATGTATTGG |
| B | B16 | 6 | 89908908 | A | T | Intergenic | C18orf1 | CEP192 | — | TATATATTATCACATACCTG |
| | | | | | | | | | | [A/T] |
| | | | | | | | | | | GTCATGTTAACTCCCTGTGA |
| B | B17 | 6 | 89909253 | T | C | Intergenic | C18orf1 | CEP192 | — | ACCACTGGTGGCAGGTGGAT |
| | | | | | | | | | | [T/C] |
| | | | | | | | | | | GACTACAAGCCTCCGAGGTG |
| B | B18 | 6 | 89909706 | C | T | Intergenic | C18orf1 | CEP192 | — | CTTCAATGTTACATATTTAA |
| | | | | | | | | | | [C/T] |
| | | | | | | | | | | TCCAGACATTTGTAAACACA |
| B | B19 | 6 | 89910103 | A | G | Intergenic | C18orf1 | CEP192 | — | GATTCACTCTGTGGCACTGT |
| | | | | | | | | | | [A/G] |
| | | | | | | | | | | TCCGGTCCCTCTGCCCCTCC |
| B | B20 | 6 | 89912254 | G | A | Intergenic | C18orf1 | CEP192 | — | TTCTTTCACAGCCTTGTGAG |
| | | | | | | | | | | [G/A] |
| | | | | | | | | | | AAGGAAAGCCTTTTCCCTTG |
| B | B21 | 6 | 89919622 | G | A | Intronic | CEP192 | — | 39 | TCTCAAAATAAATCTCTAGA |
| | | | | | | | | | | [G/A] |
| | | | | | | | | | | GATCCCATTACTAACAATAT |
| B | B22 | 6 | 89922387 | A | G | Intronic | CEP192 | — | 39 | AGCCTCCTGATGCTAAAATG |
| | | | | | | | | | | [A/G] |
| | | | | | | | | | | AAGATCCTATAATGATAAAT |
| B | B23 | 6 | 89923841 | C | T | Intronic | CEP192 | — | 39 | GGAAAAATAAGAATCTGTGA |
| | | | | | | | | | | [C/T] |
| | | | | | | | | | | TCAACAGAGTGAAAATTACA |
| B | B24 | 6 | 89924745 | G | A | Intronic | CEP192 | — | 38 | ATAACAAATTTATAAAACAA |
| | | | | | | | | | | [G/A] |
| | | | | | | | | | | TTTCTAATTATCAGGGAAAT |
| B | B25 | 6 | 89938573 | C | T | Intronic | CEP192 | — | 29 | GCATAGGCCAGCAGCTTCAG |
| | | | | | | | | | | [C/T] |
| | | | | | | | | | | TCCTATTTGACCTCTAGCCA |
| B | B26 | 6 | 89938602 | C | T | Intronic | CEP192 | — | 29 | GACCTCTAGCCAGGGAACCT |
| | | | | | | | | | | [C/T] |
| | | | | | | | | | | CATATGATGTGGATACGGCC |
| B | B27 | 6 | 89941049 | G | A | Intronic | CEP192 | — | 28 | GAATCTGAGCCCAGAGGCTG |
| | | | | | | | | | | [G/A] |
| | | | | | | | | | | AAGCACACCCTTCCTAAGTG |
| B | B28 | 6 | 89941051 | G | A | Intronic | CEP192 | — | 28 | ATCTGAGCCCAGAGGCTGAA |
| | | | | | | | | | | [G/A] |
| | | | | | | | | | | GCACACCCTTCCTAAGTGGT |
| B | B29 | 6 | 89941295 | T | C | Intronic | CEP192 | — | 28 | TGAGGTTGCAGGTTCGATCC |
| | | | | | | | | | | [T/C] |
| | | | | | | | | | | TGGCCTTCCTCAGTGGGTTA |
| B | B30 | 6 | 89941735 | A | G | Intronic | CEP192 | — | 28 | CCTGGGAACCTCCGTGTGCC |
| | | | | | | | | | | [A/G] |
| | | | | | | | | | | CAAGTGCGGCCCTAAAAAGG |
| B | B31 | 6 | 89942649 | C | T | Intronic | CEP192 | — | 27 | ACGTTGCTGTGGCTCTGGCG |
| | | | | | | | | | | [C/T] |
| | | | | | | | | | | AGGCTAGTGGCTACAGCTCC |
| B | B32 | 6 | 89942705 | T | C | Intronic | CEP192 | — | 27 | CTGGGAACCTCCATATGCCG |
| | | | | | | | | | | [T/C] |
| | | | | | | | | | | GGGAGCAGCTCAAGAAAAGA |
| B | B33 | 6 | 89943323 | C | T | Intronic | CEP192 | — | 27 | CCATAAAATTTCGTCTGACA |
| | | | | | | | | | | [C/T] |
| | | | | | | | | | | CTAGAGAATTCTGAAGTGAG |
| B | B34 | 6 | 89943764 | A | G | Intronic | CEP192 | — | 27 | AAACCTAAGAGAGAGAAAGG |
| | | | | | | | | | | [A/G] |
| | | | | | | | | | | GGAAAACCAAAGAGCCTTGT |
| B | B35 | 6 | 89945463 | C | T | Exonic | CEP192 | — | 26 | GCCAGGCACTAAGTCATTCA |
| | | | | | | | | | | [C/T] |
| | | | | | | | | | | TGTTACCATGTAACTGTCAG |
| B | B36 | 6 | 89945599 | A | C | Intronic | CEP192 | — | 25 | GTTATGCAAGCAAAGAGTAG |
| | | | | | | | | | | [A/C] |
| | | | | | | | | | | AGATTAAATTGTAGTCAGAA |
| B | B37 | 6 | 89945607 | G | A | Intronic | CEP192 | — | 25 | AGCAAAGAGTAGCAGATTAA |
| | | | | | | | | | | [G/A] |
| | | | | | | | | | | TTGTAGTCAGAAGAATATAA |
| B | B38 | 6 | 89945884 | C | A | Intronic | CEP192 | — | 25 | GGGAGTTCCCTTTGTGGCTC |
| | | | | | | | | | | [C/A] |
| | | | | | | | | | | GTGGTTAACGAACCCAACTA |
| B | B39 | 6 | 89945920 | T | C | Intronic | CEP192 | — | 25 | AACTAGGATCCATGAGGATG |
| | | | | | | | | | | [T/C] |
| | | | | | | | | | | GGGTTCAATCCCTGGCCTCA |
| B | B40 | 6 | 89946496 | T | A | Intronic | CEP192 | — | 25 | GGTTCCTCAACAAACTAAAA |
| | | | | | | | | | | [T/A] |
| | | | | | | | | | | TAGAACTACCGTATGATCCA |
| B | B41 | 6 | 89946560 | A | C | Intronic | CEP192 | — | 25 | TCCAGAGAAAACCATAAATC |
| | | | | | | | | | | [A/C] |
| | | | | | | | | | | AAAAGATACATGCACCCTTA |
| B | B42 | 6 | 89946629 | G | A | Intronic | CEP192 | — | 25 | ACCTAAATATCCATCAATAG |
| | | | | | | | | | | [G/A] |
| | | | | | | | | | | GGAATGGAAGATGTGGTACA |
| B | B43 | 6 | 89947057 | G | A | Intronic | CEP192 | — | 25 | TTTACTCTGTTGTACACCTG |
| | | | | | | | | | | [G/A] |
| | | | | | | | | | | AACTAATACAATATTATAAA |
| B | B44 | 6 | 89947199 | T | C | Intronic | CEP192 | — | 25 | CTTTCAAATTCCCAGTAATT |
| | | | | | | | | | | [T/C] |
| | | | | | | | | | | TGAATTTTAAACATGAAAAA |
| B | B45 | 6 | 89947275 | C | G | Intronic | CEP192 | — | 25 | TATAATCCTATGAATTTTGG |
| | | | | | | | | | | [C/G] |
| | | | | | | | | | | TTGCAGAGTCAGAGAGAAGT |
| B | B46 | 6 | 89947379 | G | C | Intronic | CEP192 | — | 25 | GCACGGACTCAGGCCTGAGA |
| | | | | | | | | | | [G/C] |
| | | | | | | | | | | GACCCATGCAGGTGGTGGGT |
| B | B47 | 6 | 89949591 | G | A | Intronic | CEP192 | — | 23 | TTTGTGGAAACAAGGGTCTC |
| | | | | | | | | | | [G/A] |
| | | | | | | | | | | AGTACACTGCCCAGATTTTT |
| B | B48 | 6 | 89950519 | T | A | Exonic | CEP192 | — | 23 | TGGTGGGATATCGGAGCAGC |
| | | | | | | | | | | [T/A] |
| | | | | | | | | | | AGATCCAGGTGTCAGAGACT |
| B | B49 | 6 | 89950927 | T | C | Intronic | CEP192 | — | 22 | GGCACAGCCAGTGCTCCCCC |
| | | | | | | | | | | [T/C] |
| | | | | | | | | | | GCCCCCTCCTCCCCAGGGTG |
| B | B50 | 6 | 89951556 | A | G | Intronic | CEP192 | — | 22 | CAGTGGGCTAAGGATCCGGC |
| | | | | | | | | | | [A/G] |
| | | | | | | | | | | TTGCCGTGAGCTGTGGTGTA |
| B | B51 | 6 | 89951561 | T | C | Intronic | CEP192 | — | 22 | GGCTAAGGATCCGGCGTTGC |
| | | | | | | | | | | [T/C] |
| | | | | | | | | | | GTGAGCTGTGGTGTAGGTCG |
| B | B52 | 6 | 89951607 | C | T | Intronic | CEP192 | — | 22 | GTGGCTCGGATCCCGCGTTG |
| | | | | | | | | | | [C/T] |
| | | | | | | | | | | GGTGGCTCTGGCGTAGGCCG |
| B | B53 | 6 | 89954574 | T | C | Intronic | CEP192 | — | 16 | GCTCTCAAAGACTGTGAGGC |
| | | | | | | | | | | [T/C] |
| | | | | | | | | | | GGAAGACAAACTGACAGTTT |
| B | B54 | 6 | 89956418 | A | G | Intronic | CEP192 | — | 16 | TCTGCACCAGAGAGAGAGGG |
| | | | | | | | | | | [A/G] |
| | | | | | | | | | | AGCCACATGTTACAACACTA |
| B | B55 | 6 | 89957951 | G | T | Intronic | CEP192 | — | 16 | AACCAGGATAAAAGGACATT |
| | | | | | | | | | | [G/T] |
| | | | | | | | | | | ACTGCAAAACACGGTGTCTG |
| B | B56 | 6 | 89963817 | A | C | Intronic | CEP192 | — | 13 | GCTCTCGAGTCACGAATCTA |
| | | | | | | | | | | [A/C] |
| | | | | | | | | | | ACAAAATCACCTCTCTGAAA |
| B | B57 | 6 | 89977448 | C | T | Intronic | CEP192 | — | 6 | CGGCTCGGATCCTGTGTTGC |
| | | | | | | | | | | [C/T] |
| | | | | | | | | | | GTGGCTCTGGCGTAGGCCAG |
| B | B58 | 6 | 89979528 | A | G | Intronic | CEP192 | — | 6 | CCCTGGTACCCACAAGACCC |
| | | | | | | | | | | [A/G] |
| | | | | | | | | | | GAGCTTCCTTCCCGTCACTG |
| B | B59 | 6 | 89999652 | T | C | Intergenic | CEP192 | ENSSSCG- | — | GAAGTCAATTCTTCAACCAT |
| | | | | | | | | 00000027473 | | [T/C] |
| | | | | | | | | | | CTAAGGAAATAAATTACCTA |
| B | B60 | 6 | 90042041 | C | A | Intergenic | ENSSSCG- | PTPN2 | — | CTGAGGGGACTCAGGAAACC |
| | | | | | | | 00000027473 | | | [C/A] |
| | | | | | | | | | | TATCAACAGAGTGATTCCAC |
| B | B61 | 6 | 90053901 | A | G | Intergenic | ENSSSCG- | PTPN2 | — | GCCTCCACATTTACTTACTC |
| | | | | | | | 00000027473 | | | [A/G] |
| | | | | | | | | | | ATTAACCAGTCAATAGCTCT |
| B | B62 | 6 | 90057162 | T | C | Intergenic | ENSSSCG- | PTPN2 | — | TTTTGCCAATGAAAACTCTT |
| | | | | | | | 00000027473 | | | [T/C] |
| | | | | | | | | | | CCCCAAACCATGGCGGAGTT |
| B | B63 | 6 | 90060057 | C | G | Intergenic | ENSSSCG- | PTPN2 | — | GAACACTCCCAGGTCACCAG |
| | | | | | | | 00000027473 | | | [C/G] |
| | | | | | | | | | | CTCCATTCTGACACTGTGTC |
| B | B64 | 6 | 90115462 | A | G | Intergenic | ENSSSCG- | PTPN2 | — | ATGCGGGGCACTAGGAAGCC |
| | | | | | | | 00000027473 | | | [A/G] |
| | | | | | | | | | | GTGTTCATGGGGCACTGGGC |
| B | B65 | 6 | 90121971 | A | G | Intergenic | ENSSSCG- | PTPN2 | — | AACAAAGGCAGCATGGGGGA |
| | | | | | | | 00000027473 | | | [A/G] |
| | | | | | | | | | | GGGGCAGAGTGGAGCATGGG |
| B | B66 | 6 | 90122499 | T | C | Intergenic | ENSSSCG- | PTPN2 | — | TTCAACCCCTAGCCTGGGAA |
| | | | | | | | 00000027473 | | | [T/C] |
| | | | | | | | | | | CTCCATATGCTGCAGGAGCG |
| B | B67 | 6 | 90122806 | C | A | Intergenic | ENSSSCG- | PTPN2 | — | AAAAGAAAGACCGGAAAAAA |
| | | | | | | | 00000027473 | | | [C/A] |
| | | | | | | | | | | AAACAAACAAACCAAAAATC |
| B | B68 | 6 | 90124080 | C | T | Intergenic | ENSSSCG- | PTPN2 | — | GGCTGGTTCAACTGAGGAAC |
| | | | | | | | 00000027473 | | | [C/T] |
| | | | | | | | | | | GAATTCTTAATTTCTGTCAA |
| B | B69 | 6 | 90124866 | A | C | Intergenic | ENSSSCG- | PTPN2 | — | GGTTCCTAGTCGGATTCGTT |
| | | | | | | | 00000027473 | | | [A/C] |
| | | | | | | | | | | ACCACTGCACCACGATGGGA |
| B | B70 | 6 | 90124882 | C | T | Intergenic | ENSSSCG- | PTPN2 | — | CGTTCACCACTGCACCACGA |
| | | | | | | | 00000027473 | | | [C/T] |
| | | | | | | | | | | GGGAACTCCTTATTTGCTTT |
| B | B71 | 6 | 90124893 | G | T | Intergenic | ENSSSCG- | PTPN2 | — | GCACCACGATGGGAACTCCT |
| | | | | | | | 00000027473 | | | [G/T] |
| | | | | | | | | | | ATTTGCTTTATTAAATGATT |
| B | B72 | 6 | 90127537 | A | C | Intronic | PTPN2 | — | 1 | CCGCGCGACGTGAGGGGTTC |
| | | | | | | | | | | [A/C] |
| | | | | | | | | | | CCGCCCGGGCGCTTGTTTCC |
| B | B73 | 6 | 90127616 | T | C | Intronic | PTPN2 | — | 1 | CGAGCTCTATGTTGGCTTCT |
| | | | | | | | | | | [T/C] |
| | | | | | | | | | | AGGCTACCTGCCGAGCATTT |
| B | B74 | 6 | 90131395 | T | A | Intronic | PTPN2 | — | 1 | GAAATGGCAAAAGCTAAGTC |
| | | | | | | | | | | [T/A] |
| | | | | | | | | | | TGGAGTTATGTTTCTAATAA |
| B | B75 | 6 | 90140777 | T | C | Intronic | PTPN2 | — | 1 | CCTGAAGCCCATTCTGAGTA |
| | | | | | | | | | | [T/C] |
| | | | | | | | | | | AACTGCCGAGTGTAACTGAC |
| B | B76 | 6 | 90145292 | A | G | Intronic | PTPN2 | — | 1 | AGACTCATATCAAAACTTCA |
| | | | | | | | | | | [A/G] |
| | | | | | | | | | | AATAACTACAAACCAAAAAT |
| B | B77 | 6 | 90153961 | C | T | Intronic | PTPN2 | — | 1 | CCAATGACTGTTCAGTACTA |
| | | | | | | | | | | [C/T] |
| | | | | | | | | | | GTGTGGAAATGACGCAAGGA |
| B | B78 | 6 | 90154568 | A | G | Intronic | PTPN2 | — | 1 | AAAAACAGACACAGATCAAT |
| | | | | | | | | | | [A/G] |
| | | | | | | | | | | GAACAGAGAGCTCAGAAAGA |
| B | B79 | 6 | 90157752 | T | C | Intronic | PTPN2 | — | 1 | CTCTGAACAAGGCCAGGGAT |
| | | | | | | | | | | [T/C] |
| | | | | | | | | | | GAACCCACAACCTCATGGTT |
| B | B80 | 6 | 90160707 | C | A | Intronic | PTPN2 | — | 1 | CTTCCAAGATTTAACCACTA |
| | | | | | | | | | | [C/A] |
| | | | | | | | | | | GTGTAATATTTGCTTTATGT |
| B | B81 | 6 | 90212989 | T | G | Intronic | PTPN2 | — | 1 | TACACCAGAGCCACAGCAAC |
| | | | | | | | | | | [T/G] |
| | | | | | | | | | | CGGGATCCGAGCCGAGTCTG |
| B | B82 | 6 | 90218130 | G | A | Intronic | PTPN2 | — | 2 | TATACTAGTTTACATCTGCT |
| | | | | | | | | | | [G/A] |
| | | | | | | | | | | ATCCCAAACTCCCAGTCTGC |
| B | B83 | 6 | 90218344 | A | T | Intronic | PTPN2 | — | 2 | ACTGCAAATGGCATTATTTC |
| | | | | | | | | | | [A/T] |
| | | | | | | | | | | TTTTTTTTTTTTATGGCTGG |
| B | B84 | 6 | 90218399 | T | C | Intronic | PTPN2 | — | 2 | TATTTATGTACCACATCTTC |
| | | | | | | | | | | [T/C] |
| | | | | | | | | | | ATATCTATTTCTCTGTCTAT |
| B | B85 | 6 | 90218607 | C | A | Intronic | PTPN2 | — | 2 | TGGCTGCACCAGTTTACATT |
| | | | | | | | | | | [C/A] |
| | | | | | | | | | | CTTCTATTAGCATAGAAGGG |
| B | B86 | 6 | 90218620 | C | T | Intronic | PTPN2 | — | 2 | TTACATTACTTCTATTAGCA |
| | | | | | | | | | | [C/T] |
| | | | | | | | | | | AGAAGGGTCCCCTTTTGTCT |
| B | B87 | 6 | 90248443 | T | C | Intronic | PTPN2 | — | 5 | AGCTGCATTTCCTTAAACTC |
| | | | | | | | | | | [T/C] |
| | | | | | | | | | | TGTGGTGCTCAGAAAAGTCT |
| B | B88 | 6 | 90251336 | C | G | Intronic | PTPN2 | — | 5 | GGGTGATGTGGGTGTGGGCT |
| | | | | | | | | | | [C/G] |
| | | | | | | | | | | GGGTGGGTGTCCCAGGAGGG |
| B | B89 | 6 | 90254945 | A | G | Intronic | PTPN2 | — | 5 | GGAGGGATTGGAATGGACTA |
| | | | | | | | | | | [A/G] |
| | | | | | | | | | | GAATTTGGGGTCAATAGATG |
| B | B90 | 6 | 90293922 | A | C | Intergenic | PTPN2 | ENSSSCG- | — | AGGCCACGCCACATGCTGGA |
| | | | | | | | | 00000021827 | | [A/C] |
| | | | | | | | | | | CTGGTTGCGAACATGGGTCT |
| B | B91 | 6 | 90311616 | A | T | Intergenic | ENSSSCG- | ENSSSCG- | — | GGAACAATGAGGTTGCGGGT |
| | | | | | | | 00000021827 | 00000029671 | | [A/T] |
| | | | | | | | | | | CAATCCCTGCCCTTGCTCAC |
| C | C1 | 7 | 63714553 | A | G | Intergenic | UBL7 | ENSSSCG- | — | GGCAGAGATTTGCAAGAGCA |
| | | | | | | | | 00000001905 | | [A/G] |
| | | | | | | | | | | TTGGACAAACAGAACTGCAT |
| D | D1 | 15 | 51799437 | T | C | Intergenic | C4ORF41 | STOX2 | — | CCTTGGTGAGTTTGTCCATC |
| | | | | | | | | | | [T/C] |
| | | | | | | | | | | CCAAACATTGCTACCTTTGG |
| D | D2 | 15 | 51800022 | T | C | Intergenic | C4ORF41 | STOX2 | — | GTAACTAGCAGTTTGGAGCT |
| | | | | | | | | | | [T/C] |
| | | | | | | | | | | GGGCAAAGGAGAACAAAACA |
| D | D3 | 15 | 51800356 | T | G | Intergenic | C4ORF41 | STOX2 | — | TTATATTTCTGGAAGAAGCA |
| | | | | | | | | | | [T/G] |
| | | | | | | | | | | AAAGATGACTATCAGACACA |
| E | E1 | 3 | 72655441 | T | C | Intergenic | ALMS1 | EGR4 | — | TTTTATACTTCTTTCCACTC |
| | | | | | | | | | | [T/C] |
| | | | | | | | | | | CTGACTTTCAGTACATGATA |
| E | E2 | 3 | 72655561 | T | C | Intergenic | ALMS1 | EGR4 | — | TTCCTTTTTCTTTTTAGAAC |
| | | | | | | | | | | [T/C] |
| | | | | | | | | | | GCACTTTCGGCACGTGGAAG |
| E | E3 | 3 | 72655574 | C | T | Intergenic | ALMS1 | EGR4 | — | TTAGAACTGCACTTTCGGCA |
| | | | | | | | | | | [C/T] |
| | | | | | | | | | | GTGGAAGTTTCCAGGCTAGG |
| E | E4 | 3 | 72657516 | T | C | Intergenic | ALMS1 | EGR4 | — | GCAAACGTCCGTACTACCGC |
| | | | | | | | | | | [T/C] |
| | | | | | | | | | | AGAGTGTTGGAAGACTGAAT |
| E | E5 | 3 | 72704273 | T | C | Intergenic | EGR4 | FBXO41 | — | TTCTTGATTGCTCTCAGATC |
| | | | | | | | | | | [T/C] |
| | | | | | | | | | | ATCCTTTCCCAAATTTGCAC |
| E | E6 | 3 | 72704713 | A | G | Intergenic | EGR4 | FBXO41 | — | CAAACACCTACTGAGCACCT |
| | | | | | | | | | | [A/G] |
| | | | | | | | | | | CCCCATGACGAGCACTGTTG |
| E | E7 | 3 | 72737256 | A | G | Intronic | CCT7 | ENSSSCT- | 6 | AGGATCAGAAAAGGTCCAAA |
| | | | | | | | | 00000009094 | | [A/G] |
| | | | | | | | | | | CAGCACAGAGACACCAACAT |
| E | E8 | 3 | 72740541 | T | A | Intronic | CCT7 | ENSSSCT- | 3 | CCTAAAATATGAAATCATAC |
| | | | | | | | | 00000009094 | | [T/A] |
| | | | | | | | | | | TGTAAATTAACTGAGAACAT |
| E | E9 | 3 | 72746539 | A | G | Intergenic | CCT7 | PRADC1 | — | ACCTGCACTAGAGCCGTGAC |
| | | | | | | | | | | [A/G] |
| | | | | | | | | | | CCAGATCCTTAACCACTAGG |
| E | E10 | 3 | 72759645 | T | C | Intronic | SMYD5 | ENSSSCT- | 9 | AGGCCAAGGCTGCATTTCAC |
| | | | | | | | | 00000009096 | | [T/C] |
| | | | | | | | | | | AACCTGCCTCGATGTCCTTG |
| E | E11 | 3 | 72761468 | T | C | Intronic | SMYD5 | ENSSSCT- | 5 | TGCCCTCAGAGAAACACCAT |
| | | | | | | | | 00000009096 | | [T/C] |
| | | | | | | | | | | GCACAACAGCTTCTAGGGAG |
| E | E12 | 3 | 72765464 | G | A | Intronic | SMYD5 | ENSSSCT- | 1 | GTGCATGTTAAGTGAGCGGT |
| | | | | | | | | 00000009096 | | [G/A] |
| | | | | | | | | | | CTGGTCTAAAGCACTGTGTA |
| E | E13 | 3 | 72765797 | G | C | Intronic | SMYD5 | ENSSSCT- | 1 | GCAGGATGTGTAGACCTGGA |
| | | | | | | | | 00000009096 | | [G/C] |
| | | | | | | | | | | AGGCCCTGAGCAACCCATGA |
| E | E14 | 3 | 72765861 | A | G | Intronic | SMYD5 | ENSSSCT- | 1 | CAACTCTGGAGGCTCAAACA |
| | | | | | | | | 00000009096 | | [A/G] |
| | | | | | | | | | | TAGTGTGAGACAGCTGGAAC |
| E | E15 | 3 | 72769594 | C | T | Intergenic | SMYD5 | NOTO | — | GGGGTCTTTGGACCCAGCTA |
| | | | | | | | | | | [C/T] |
| | | | | | | | | | | GGGTTAGAGAGCAATAGGCT |
| E | E16 | 3 | 72774789 | T | C | Intergenic | NOTO | RAB11 | — | TTTGAAAAAGAAGCCCTGGC |
| | | | | | | | | FIP5 | | [T/C] |
| | | | | | | | | | | ACACTTGCCTGTGACTACGC |
| E | E17 | 3 | 72784289 | C | T | Intergenic | NOTO | RAB11 | — | CCACCCACCTGGCTCCCTAA |
| | | | | | | | | FIP5 | | [C/T]TGTGTCTTTATTTTTATTTT |
| E | E18 | 3 | 72786328 | G | A | Intergenic | NOTO | RAB11 | — | CCATTTCTACTAAAAAAGGA |
| | | | | | | | | FIP5 | | [G/A] |
| | | | | | | | | | | GTTCAGAGGAACTCAGGGAC |
| E | E19 | 3 | 72790350 | G | A | Intergenic | NOTO | RAB11 | — | AATGAATGGGAGATGGAGTT |
| | | | | | | | | FIP5 | | [G/A] |
| | | | | | | | | | | TAGTAGAAGAGTGTGTTTGA |
| E | E20 | 3 | 72790678 | G | A | Intergenic | NOTO | RAB11 | — | GAACCATGAGGTGCAGGTTC |
| | | | | | | | | FIP5 | | [G/A] |
| | | | | | | | | | | ATCCCTGGCCTCGCTCAGTG |
| E | E21 | 3 | 72790778 | A | G | Intergenic | NOTO | RAB11 | — | CACGGTGCTGTGGCTCTGGC |
| | | | | | | | | FIP5 | | [A/G] |
| | | | | | | | | | | AAGGCGGCAGCTACAGCTCC |
| E | E22 | 3 | 72795642 | G | A | Intergenic | NOTO | RAB11 | — | TCCTTTTGCCAATTCCCCTA |
| | | | | | | | | FIP5 | | [G/A] |
| | | | | | | | | | | TAAATCTTTCTCTTGTGTAT |
| E | E23 | 3 | 72795872 | T | C | Intergenic | NOTO | RAB11 | — | TGGACCACCAGGAAACTCCC |
| | | | | | | | | FIP5 | | [T/C] |
| | | | | | | | | | | TGTATATAAGTTTTTAATTT |

TABLE 2
Associations of the SNP sites on Chromosome 6 with SNP A2

| Chromosome | Position | r² | P value (χ², df = 1) |
|---|---|---|---|
| 6 | 122097788 | 0.947 | 0.00E+00 |
| 6 | 122113635 | | |
| 6 | 122123140 | 0.806 | 0.00E+00 |
| 6 | 122125228 | 0.806 | 0.00E+00 |
| 6 | 122126620 | 0.858 | 0.00E+00 |
| 6 | 122126799 | 0.849 | 0.00E+00 |
| 6 | 122148636 | 0.844 | 0.00E+00 |
| 6 | 122178848 | 0.849 | 0.00E+00 |
| 6 | 122178862 | 0.849 | 0.00E+00 |
| 6 | 122178870 | 0.947 | 0.00E+00 |
| 6 | 122197541 | 0.895 | 0.00E+00 |
| 6 | 122197588 | 0.947 | 0.00E+00 |
| 6 | 122217096 | 0.947 | 0.00E+00 |

TABLE 3
Associations of the SNP sites on Chromosome 6 with SNP B13

| Chromosome | Position | r² | P value (χ², df = 1) |
|---|---|---|---|
| 6 | 89403626 | 0.812 | 0 |
| 6 | 89412355 | 0.898 | 0 |
| 6 | 89412733 | 0.898 | 0 |
| 6 | 89413181 | 0.898 | 0 |
| 6 | 89413302 | 0.8 | 0 |
| 6 | 89413372 | 0.898 | 0 |
| 6 | 89413471 | 0.898 | 0 |
| 6 | 89413560 | 0.812 | 0 |
| 6 | 89414704 | 0.812 | 0 |
| 6 | 89414733 | 0.812 | 0 |
| 6 | 89431138 | 0.898 | 0 |
| 6 | 89888614 | 1 | 0 |
| 6 | 89899151 | | |
| 6 | 89902460 | 1 | 0 |
| 6 | 89906636 | 0.947 | 0 |
| 6 | 89908908 | 0.947 | 0 |
| 6 | 89909253 | 0.8 | 0 |
| 6 | 89909706 | 0.898 | 0 |
| 6 | 89910103 | 0.854 | 0 |
| 6 | 89912254 | 0.945 | 0 |
| 6 | 89919622 | 0.945 | 0 |
| 6 | 89922387 | 0.838 | 0 |
| 6 | 89923841 | 0.947 | 0 |
| 6 | 89924745 | 0.891 | 0 |
| 6 | 89938573 | 0.8 | 0 |
| 6 | 89938602 | 0.854 | 0 |
| 6 | 89941049 | 1 | 0 |
| 6 | 89941051 | 1 | 0 |
| 6 | 89941295 | 0.891 | 0 |
| 6 | 89941735 | 0.812 | 0 |
| 6 | 89942649 | 0.838 | 0 |
| 6 | 89942705 | 1 | 0 |
| 6 | 89943323 | 0.8 | 0 |
| 6 | 89943764 | 0.947 | 0 |
| 6 | 89945463 | 1 | 0 |
| 6 | 89945599 | 0.898 | 0 |
| 6 | 89945607 | 1 | 0 |
| 6 | 89945884 | 0.838 | 0 |
| 6 | 89945920 | 1 | 0 |
| 6 | 89946496 | 0.891 | 0 |
| 6 | 89946560 | 0.854 | 0 |
| 6 | 89946629 | 0.892 | 0 |
| 6 | 89947057 | 1 | 0 |
| 6 | 89947199 | 1 | 0 |
| 6 | 89947275 | 0.891 | 0 |
| 6 | 89947379 | 0.838 | 0 |
| 6 | 89949591 | 1 | 0 |
| 6 | 89950519 | 0.945 | 0 |
| 6 | 89950927 | 0.891 | 0 |
| 6 | 89951556 | 0.945 | 0 |
| 6 | 89951561 | 1 | 0 |
| 6 | 89951607 | 0.898 | 0 |
| 6 | 89954574 | 0.945 | 0 |
| 6 | 89956418 | 0.812 | 0 |
| 6 | 89957951 | 0.892 | 0 |
| 6 | 89963817 | 1 | 0 |
| 6 | 89977448 | 0.891 | 0 |
| 6 | 89979528 | 0.945 | 0 |
| 6 | 89999652 | 1 | 0 |
| 6 | 90042041 | 1 | 0 |
| 6 | 90053901 | 0.891 | 0 |
| 6 | 90057162 | 0.892 | 0 |
| 6 | 90060057 | 0.838 | 0 |
| 6 | 90115462 | 0.945 | 0 |
| 6 | 90121971 | 0.891 | 0 |
| 6 | 90122499 | 0.947 | 0 |
| 6 | 90122806 | 1 | 0 |
| 6 | 90124080 | 0.898 | 0 |
| 6 | 90124866 | 0.838 | 0 |
| 6 | 90124882 | 0.898 | 0 |
| 6 | 90124893 | 1 | 0 |
| 6 | 90127537 | 0.892 | 0 |
| 6 | 90127616 | 0.838 | 0 |
| 6 | 90131395 | 0.891 | 0 |
| 6 | 90140777 | 0.898 | 0 |
| 6 | 90145292 | 1 | 0 |
| 6 | 90153961 | 0.898 | 0 |
| 6 | 90154568 | 0.898 | 0 |
| 6 | 90157752 | 1 | 0 |
| 6 | 90160707 | 0.898 | 0 |
| 6 | 90212989 | 0.898 | 0 |
| 6 | 90218130 | 1 | 0 |
| 6 | 90218344 | 1 | 0 |
| 6 | 90218399 | 1 | 0 |
| 6 | 90218607 | 0.898 | 0 |
| 6 | 90218620 | 0.898 | 0 |
| 6 | 90248443 | 0.891 | 0 |
| 6 | 90251336 | 0.898 | 0 |
| 6 | 90254945 | 0.844 | 0 |
| 6 | 90293922 | 0.8 | 0 |
| 6 | 90311616 | 0.891 | 0 |

TABLE 4
Associations of the SNP sites on Chromosome 15 with SNP D1

| Chromosome | Position | r² | P value (χ², df = 1) |
|---|---|---|---|
| 15 | 51799437 | | |
| 15 | 51800022 | 0.915 | 0 |
| 15 | 51800356 | 0.876 | 0 |

TABLE 5
Associations of the SNP sites on Chromosome 3 with SNP E10

| Chromosome | Position | r² | P value (χ², df = 1) |
|---|---|---|---|
| 3 | 72655441 | 1 | 2.73E−06 |
| 3 | 72655561 | 1 | 2.73E−06 |
| 3 | 72655574 | 1 | 2.73E−06 |
| 3 | 72657516 | 1 | 2.73E−06 |
| 3 | 72704273 | 1 | 2.73E−06 |
| 3 | 72704713 | 1 | 2.73E−06 |
| 3 | 72737256 | 1 | 2.73E−06 |
| 3 | 72740541 | 1 | 2.73E−06 |
| 3 | 72746539 | 1 | 2.73E−06 |
| 3 | 72759645 | | |
| 3 | 72761468 | 1 | 2.73E−06 |
| 3 | 72765464 | 1 | 2.73E−06 |
| 3 | 72765797 | 1 | 2.73E−06 |
| 3 | 72765861 | 1 | 2.73E−06 |
| 3 | 72769594 | 1 | 2.73E−06 |
| 3 | 72774789 | 1 | 2.73E−06 |
| 3 | 72784289 | 1 | 2.73E−06 |
| 3 | 72786328 | 1 | 2.73E−06 |
| 3 | 72790350 | 1 | 2.73E−06 |
| 3 | 72790678 | 1 | 2.73E−06 |
| 3 | 72790778 | 1 | 2.73E−06 |
| 3 | 72795642 | 1 | 2.73E−06 |
| 3 | 72795872 | 1 | 2.73E−06 |

1. A method of identifying a pig as producing an increased litter size, comprising:
detecting for presence or absence of an E allele SNP marker in at least one single nucleotide polymorphism (SNP) site within a genomic region selected from Sweep A, Sweep B, Sweep C, Sweep D, and Sweep E, in a pig genome,
wherein the presence of the E allele SNP marker indicates an increased litter size of the pig.

2. The method of claim 1, wherein the genomic region is Sweep A.

3. The method of claim 2, further comprising detecting for presence or absence of the E allele SNP marker in the SNP site of one or both copies of chromosomes.

4. The method of claim 3, wherein the presence of the E allele SNP marker in the SNP site of one or both copies of chromosomes indicates an increased litter size of the pig.

5. The method of claim 1, wherein the genomic region is Sweep B.

6. The method of claim 5, further comprising detecting for presence or absence of the E allele SNP marker in the SNP site of both copies of chromosomes.

7. The method of claim 6, wherein the presence of the E allele SNP marker in the SNP site of both copies of chromosomes indicates an increased litter size of the pig.

8. The method of claim 1, wherein the genomic region is Sweep C.

9. The method of claim 8, further comprising detecting for presence or absence of the E allele SNP marker in the SNP site of one or both copies of chromosomes.

10. The method of claim 9, wherein the presence of the E allele SNP marker in the SNP site of one or both copies of chromosomes indicates an increased litter size of the pig.

11. The method of claim 1, wherein the genomic region is Sweep D.

12. The method of claim 11, further comprising detecting for presence or absence of the E allele SNP marker in the SNP site of both copies of chromosomes.

13. The method of claim 12, wherein the presence of the E allele SNP marker in the SNP site of one copy of chromosomes indicates an increased litter size of the pig.

14. The method of claim 1, wherein the genomic region is Sweep E.

15. The method of claim 14, further comprising detecting for presence or absence of the E allele SNP marker in the SNP site of one or both copies of chromosomes.

16. The method of claim 15, wherein the presence of the E allele SNP marker in the SNP site of one or both copies of chromosomes indicates an increased litter size of the pig.

17. The method of claim 1, further comprising selecting the pig for breeding if the pig is identified as producing an increased litter size.

18. The method of claim 1, wherein the Sweep A genomic region spans from Chr6: 122097788 to Chr6: 122217096 (NCBI build Sscrofa 10.2), the Sweep B genomic region spans from Chr6: 89403626 to Chr6: 90311616 (NCBI build Sscrofa 10.2), the Sweep C genomic region locates at Chr7: 63714553 (NCBI build Sscrofa 10.2), the Sweep D genomic region spans from Chr15: 51799437 to Chr15: 51800356 (NCBI build Sscrofa 10.2), and the Sweep E genomic region spans from Chr3: 72655441 to Chr3: 72795872 (NCBI build Sscrofa 10.2).

19. The method of claim 1, wherein the at least one SNP site within Sweep A genomic region is selected from the SNPs: A1-A13 as listed in Table 1, the at least one SNP site within Sweep B genomic region is selected from the SNPs: B1-B91 as listed in Table 1, the SNP site within Sweep C genomic region is SNP C1 as listed in Table 1, the at least one SNP site within Sweep D genomic region is selected from the SNPs: D1-D3 as listed in Table 1, and the at least one SNP site within Sweep E genomic region is selected from the SNPs: E1-E23 as listed in Table 1.

20. The method of claim 1, wherein the at least one SNP site within Sweep A genomic region comprises SNP A2, the at least one SNP site within Sweep B genomic region comprises SNP B13, the SNP site within Sweep C genomic region comprises SNP C1, the at least one SNP site within Sweep D genomic region comprises SNP D1, and the at least one SNP site within Sweep E genomic region comprises SNP E10.

21. The method of claim 1, wherein the at least one SNP site within Sweep A genomic region is in linkage disequilibrium with SNP A2, the at least one SNP site within Sweep B genomic region is in linkage disequilibrium with SNP B13, the at least one SNP site within Sweep D genomic region is in linkage disequilibrium with SNP D1, and the at least one SNP site within Sweep E genomic region is in linkage disequilibrium with SNP E10.

22. The method of claim 1, wherein the E allele SNP marker is the high-frequency SNP marker at the corresponding SNP site in an Erhualian pig genome.

23. The method of claim 1, wherein the E allele SNP marker is as shown in Table 1.

24. The method of any of claim 1, wherein the pig is a sow.

25. The method of claim 1, wherein the pig is an offspring of a Taihu pig.

26. The method of claim 1, wherein the pig is an offspring of an Erhualian pig.

27. The method of claim 1, wherein the detecting comprises sequencing at least a nucleic acid fragment containing the SNP site in a nucleic acid sample from the pig.

28. The method of claim 1, wherein the detecting comprises detecting an amplification product of at least a nucleic acid fragment containing the SNP site in a nucleic acid sample from the pig.

29. The method of claim 1, wherein the detecting comprises detecting hybridization of a probe to at least a nucleic acid fragment containing the SNP site in a nucleic acid sample from the pig.

30. The method of claim 1, wherein the detecting comprises detecting a primer extension product of at least a nucleic acid fragment containing the SNP site in a nucleic acid sample from the pig.

31. The method of claim 1, wherein the detecting comprises detecting restriction digestion product of at least a nucleic acid fragment containing the SNP site in a nucleic acid sample from the pig.

32. The method of claim 1, wherein the detecting comprises detecting gel electrophoresis results of at least a nucleic acid fragment containing the SNP site in a nucleic acid sample from the pig.

33. The method of claim 1, wherein the detecting comprises detecting binding affinity of a protein to at least a nucleic acid fragment containing the SNP site in a nucleic acid sample from the pig.

34. A method of detecting the SNP marker at a SNP site within Sweep A region in a pig, comprising:
determining the presence or absence of an E allele SNP marker or an O allele SNP marker in at least one single nucleotide allele (SNP) site within Sweep A genomic region in a pig genome.

35. The method of claim 34, wherein the Sweep A genomic region spans from Chr6: 122097788 to Chr6: 122217096 (NCBI build Sscrofa 10.2).

36. The method of claim 35, wherein the at least one SNP site within Sweep A genomic region is selected from the SNPs: A1-A13 as listed in Table 1.

37. A method of detecting the SNP marker at a SNP site within Sweep B region in a pig, comprising:
determining the presence or absence of an E allele SNP marker or an O allele SNP marker in at least one single nucleotide allele (SNP) site within Sweep B genomic region in a pig genome.

38. The method of claim 37, wherein the Sweep B genomic region spans from Chr6: 89403626 to Chr6: 90311616 (NCBI build Sscrofa 10.2).

39. The method of claim 35, wherein the at least one SNP site within Sweep B genomic region is selected from the SNPs: B1-B91 as listed in Table 1.

40. A method of detecting the SNP marker at a SNP site within Sweep C region in a pig, comprising:
determining the presence or absence of an E allele SNP marker or an O allele SNP marker in the single nucleotide allele (SNP) site within Sweep C genomic region in a pig genome.

41. The method of claim 40, wherein the Sweep C genomic region locates at Chr7: 63714553 (NCBI build Sscrofa 10.2).

42. The method of claim 41, wherein the SNP site within Sweep C genomic region is SNP C1 as listed in Table 1.

43. A method of detecting the SNP marker at a SNP site within Sweep D region in a pig, comprising:
determining the presence or absence of an E allele SNP marker or an O allele SNP marker in at least one single nucleotide allele (SNP) site within Sweep D genomic region in a pig genome.

44. The method of claim 43, wherein the Sweep D genomic region spans from Chr15: 51799437 to Chr15: 51800356 (NCBI build Sscrofa 10.2).

45. The method of claim 44, wherein the at least one SNP site within Sweep D genomic region is selected from the SNPs: D1-D3 as listed in Table 1.

46. A method of detecting the SNP marker at a SNP site within Sweep E region in a pig, comprising:
determining the presence or absence of an E allele SNP marker or an O allele SNP marker in at least one single nucleotide allele (SNP) site within Sweep E genomic region in a pig genome.

47. The method of claim 43, wherein the Sweep E genomic region spans from Chr3: 72655441 to Chr3: 72795872 (NCBI build Sscrofa 10.2).

48. The method of claim 47, wherein the at least one SNP site within Sweep D genomic region is selected from the SNPs: E1-E23 as listed in Table 1.

49. An isolated oligonucleotide primer, useful in selectively amplifying a polynucleotide fragment containing or lacking an E allele SNP marker in at least one of the SNP sites listed in Table 1.

50. (canceled)

51. An isolated oligonucleotide probe useful in selectively hybridizing to a polynucleotide fragment containing or lacking an E allele SNP marker in one or more SNP sites listed in Table 1.

52. (canceled)

53. A kit useful in the method of claim 1, comprising the isolated oligonucleotide primer of claim 49, or the isolated oligonucleotide probe of claim 51.
